(19) United States
(12) Patent Application Publication
(10) Pub. No.: US 2023/0293469 A1
(43) Pub. Date: Sep. 21, 2023

(54) NOVEL PHARMACEUTICAL COMPOSITIONS

(71) Applicant: NEUROPRO THERAPEUTICS, INC., Yachats, OR (US)

(72) Inventor: Daryl W. HOCHMAN, Yachats, OR (US)

(21) Appl. No.: 18/013,697

(22) PCT Filed: Jul. 1, 2021
(86) PCT No.: PCT/US21/40084

§ 371 Date: Dec. 29, 2022

Related U.S. Application Data
(60) Provisional application No. 63/046,743, filed on Jul. 1, 2020.

(51) Int. Cl. A61K 31/196; A61K 45/06

(52) U.S. Cl. CPC A61K 31/196; A61K 45/06

(57) ABSTRACT

The present disclosure relates to novel pharmaceutical compositions for active pharmaceutical ingredients subject to first pass metabolism. More particularly, bumetanide analogs, including bumetanide dibenzylamide, bumetanide diethylamide, and bumetanide morpholinoamide, are demonstrated as susceptible to first pass metabolism, thereby prompting effort toward developing an acceptable pharmaceutical formulation to provide adequate bioavailability.

### CROSS-REFERENCE TO RELATED APPLICATIONS

The present application claims the benefit of U.S. Provisional Patent Application Ser. No. 63/046,743, filed Jul. 1, 2020, which is herein incorporated by reference in its entirety.

### FIELD OF THE INVENTION

The present disclosure relates to novel pharmaceutical compositions for active pharmaceutical ingredients subject to first pass metabolism. More particularly, bumetanide analogs, including bumetanide dibenzylamide, bumetanide diethylamide, and bumetanide morpholinoamide, are demonstrated as susceptible to first pass metabolism, thereby prompting effort toward developing an acceptable pharmaceutical formulation.

### BACKGROUND OF THE INVENTION

The Sodium-Potassium-Chloride co-transporter (NKCC) is a membrane transporter protein that moves sodium, potassium, and chloride into cells to help maintain transmembrane ion gradients (Russell J. M., 2000). There are two isoforms of this co-transporter found in humans, NKCC1 and NKCC2. NKCC2 is renal specific, found only in the kidney on the thick ascending limb of the loop of Henle. NKCC1 is found in most types of cells and tissues (Jackson, 2018), including neurons and glial cells (Loscher, Puskarjov, & Kaila, 2013).

NKCC antagonists, such as furosemide (LASIX®) and bumetanide (BUMEX®), have been used clinically for decades for the management of edema, and have well established safety profiles (Wargo & Banta, 2009). In the 1990's, it was discovered that NKCC antagonists also have potent anti-seizure effects in laboratory seizure models (Hochman, Baraban, Owens, & Schwartzkroin, 1995; Schwartzkroin, Baraban, & Hochman, 1998). NKCC1 presents a novel pharmacological target for treatment of epilepsy.

Two sets of observations from these studies suggested that NKCC antagonists might be more efficacious with fewer CNS side effects than existing antiepileptic drugs (AEDs): NKCC antagonists completely blocked seizure activity across a variety of different laboratory seizure models, where seizure activity had been elicited by a spectrum of different synaptic and non-synaptic mechanisms. These drugs also blocked seizures in several models that are thought to be resistant to all existing (at the time of the studies) AEDs. These results suggest that NKCC antagonists might be more broadly therapeutic for a larger number of epilepsy disorders than are current AEDs, and they might possibly be efficacious for some patients who are unable to adequately control their seizures with any of the currently available AEDs. These possibilities are supported by data from several small clinical studies suggesting that NKCC antagonists reduce seizure frequency in patients suffering from medically intractable epilepsy (Ahmad, Clarke, Hewett, & Richens, 1976; Haglund & Hochman, 2005; Eftekhari, et al., 2013; Gharaylou, et al., 2019).

The blockade of seizure activity by NKCC antagonists occurs independent of any suppression or modulation of neuronal excitability or synaptic transmission. These drugs appear to block the “hypersynchrony” that defines electrographic seizure activity, without affecting neuronal or synaptic excitability (Hochman, Baraban, Owens, & Schwartzkroin, 1995). This is a distinctly different mechanism of action than currently used AEDs that are thought to mediate their antiepileptic effects through suppressing the excitability of brain tissue, through some combination of blocking ion channels on neurons, increasing synaptic inhibition, and decreasing synaptic excitation (Sills & Rogawski, 2020). This suggests that NKCC antagonists might avoid the neurological and cognitive side effects that are associated with some of the currently prescribed AEDs (Barr, 2019). This is important, since it is estimated that 30%-50% of epilepsy patents are not adherent to their drug regimen because of side effects (McAuley, et al., 2015), and decreased AED adherence is associated with a more than a 3-fold increase in mortality (Faught, Duh, Weiner, Guérin, & Cunnington, 2008).

There is a need for novel treatment approaches to seizure disorders, especially an approach that avoids a negative side effect profile.

### SUMMARY OF THE INVENTION

One rationale for addressing the present need includes developing prodrugs and analogs of bumetanide to treat epilepsy.

The characteristics of an NKCC antagonist that should be optimized to treat epilepsy include: 1) a lower selectivity for the renal-specific NKCC2 over the NKCC1 isoforms to limit diuretic effects, 2) a high affinity for the NKCC1 isoform to optimize its antiseizure effects, and 3) a high affinity for crossing the blood-brain-barrier (B-B-B) to increase its concentration in cerebral tissue. However, none of the existing NKCC antagonists adequately satisfy all three properties. First, all existing NKCC antagonists show equal selectivity for the NKCC1 and NKCC2 isoforms (Hannaert, Alvarez-Guerra, Pirot, Nazaret, & Garay, 2002), and for the two splice variants of NKCC1 (Hampel, Romermann, MacAulay, & Loscher, 2018). Second, of the six NKCC antagonists that have been used clinically in the United States or Europe, bumetanide has the greatest potency by 40× (Jackson, 2018). Hence, not only would bumetanide be predicted be a significantly more efficacious antiepileptic agent than the other NKCC antagonists via NKCC1 antagonism, but it is also most potently diuretic via NKCC2 antagonism. Third, the existing NKCC antagonists, and specifically bumetanide, have poor brain penetration (Li, et al., 2011). This is unsurprising, since NKCC antagonists were optimized for their diuretic effects, which favored selection of compounds that antagonized NKCC2 cotransporters within kidney (Jackson, 2018), rather than for an ability to cross the B-B-B.

A classic medicinal chemistry approach for improving the physicochemical properties of a molecule with known therapeutic CNS effects, to better cross the B-B-B and reduce systemic side effects, is to create “prodrugs” of that structure (Zeiadeh, Najjar, & Karaman, 2018; Rautio, Laine, Gynther, & Savolainen, 2008). In this context, a CNS prodrug is a molecule that is inactive in the form in which it is administered, and becomes converted into the active intended drug once hydrolyzed by enzymes in the brain or tissues that comprise the B-B-B.

Bumetanide is a compelling candidate for the prodrug approach. By attaching amides, esters, or other chemical groups at various sites on the bumetanide molecule, one hopes to make it inactive systemically, thus reducing its diuretic effects, and more lipophilic so that it more easily crosses the blood-brain barrier, where it can be enzymatically converted into active bumetanide, thus increasing its efficacy. Bumetanide in solution is 99% negatively charged at pH 7.4 (Lykke, et al., 2015). Because bumetanide is ionized into a negatively charged species at the brain pH, the maintenance of a therapeutic concentration in the CNS is expected to be prolonged (Rautio, Laine, Gynther, & Savolainen, 2008).

A bumetanide prodrug approach, along with supportive data showing enhanced CNS effects, was first reported in a patent with the United States Trademark and Patent Office (Hochman & Partridge, 2011). There has been subsequent validation of the bumetanide prodrug approach, in animal seizure models, by several different academic research groups (Auer, Schreppel, Erker, & Schwarzer, 2019; Töllner, et al., 2014). One approach has been to focus on the development of a molecule in which a prodrug structure has been added to bumetanide. Amide analogs of bumetanide, such as this one, are presumably hydrolyzed by peptidases and amidases in the B-B-B and brain (Brownlees & Williams, 1993), where it would be converted back into bumetanide.

Certain bumetanide prodrugs are disclosed in U.S. Pat. No. 8,008,283, hereby incorporated by reference in its entirety. Particular compounds identified as active agents in the present disclosure include Example 7 of US '283, bumetanide diethylamide and Example 8 of US '283, bumetanide dibenzylamide. In one embodiment of the present disclosure, a preferred active agent is bumetanide dibenzylamide. Other bumetanide prodrugs include bumetanide N-morpholinoamide, disclosed as Example 125 of US 2017/0246131.

When given orally, a drug is absorbed into the enterocyte monolayer in the basolateral side of the intestine, where it can undergo metabolism and/or efflux back into the lumen by trans-membranal transporters. From the apical side of the entrocytes the drug is delivered via the portal vein to the liver and thereafter into the systemic blood circulation. Bioavailability is defined as the fraction of an administered dose of unchanged drug that reaches the systemic circulation. By definition, when a medication is administered intravenously, its bioavailability is 100%.

Research in the field of drug absorption has focused on ways to increase drug efficacy by increasing drug absorption. To this end, methods have been used to increase drug absorption using liposomes as carriers and by designing more lipophilic drugs. However, these methods have not been successful in circumventing liver biotransformation and biliary secretion of drugs.

Thus, when a medication is orally administered, its bioavailability generally decreases due to incomplete absorption and first-pass metabolism and also may vary from patient to patient. Bioavailability is one of the essential tools in pharmacokinetics, as it must be considered when calculating dosages for none intravenous routes of administration.

Despite the great advancements in the area of various drug delivery systems such as nano-lipospheres, many drugs are prone to poor oral bioavailability due to biological barriers at the enterocyte level, termed “intestinal first pass metabolism.” These biological processes include Phase I metabolism, namely oxidative enzymes, and Phase II metabolism including conjugation, sulphation and glucuronidation by intestinal enzymes. In addition, the poor oral bioavailability is attributed to efflux transporters, e.g., permeability-glycoprotein (P-gp) at the enterocyte luminal membrane.

Surprisingly, and as demonstrated herein, the compounds identified as active agents in the present disclosure, exhibit poor oral bioavailability. As discussed in more detail, in at least one species, oral bioavailability of the compounds identified as active agent in the present disclosure is approximately less than 50%, 45%, 40%, 35%, 30%, 25%, 20%, 15%, 10%, 9%, 8%, 7%, 6%, 5%, 4%, 3%, 2%, 1% or approaches 0%.

Only upon identification of the poor bioavailability of the compounds identified as active agents in the present disclosure did the present inventors endeavor to remediate the substantial first pass metabolic effects.

One embodiment of the present disclosure includes a pharmaceutical composition comprising an active agent selected from one or more of bumetanide morpholinoamide, bumetanide diethylamide, and bumetanide dibenzylamide, or a salt thereof; and one or more excipient to increase bioavailability of the one or more active agent. In one aspect of the present disclosure, the active agent is bumetanide dibenzylamide.

One embodiment of the present disclosure includes a pharmaceutical composition comprising: an active agent bumetanide dibenzylamide, or a salt thereof and one or more excipient, wherein the minimum area under curve is 57 ng*h/mL for each immediate release formulated dose, and a minimum total AUC of 114 ng*h/mL over a 24 hr period with a sustained release formulated dose.

One embodiment of the present disclosure includes a pharmaceutical composition comprising an active agent bumetanide morpholinoamide, or a salt thereof and one or more excipient, wherein the minimum area under curve is 45 ng*h/mL for each immediate release formulated dose, and a minimum total AUC of 84 ng*h/mL over a 24 hr period with a sustained release formulated dose.

One embodiment of the present disclosure includes a pharmaceutical composition comprising an active agent bumetanide diethylamide, or a salt thereof and one or more excipient, wherein the minimum area under curve is 42 ng*h/mL for each immediate release formulated dose, and a minimum total AUC of 84 ng*h/mL over a 24 hr period with a sustained release formulated dose.

In one aspect, the composition is selected from the group consisting of: a sublingual tablet, a sublingual amorphous solid dispersion, an orodispersible film, a fast dissolving film, a nanoparticle spray, a muco-adhesion patch, a liquid, a slick pack, a lipid-filled soft gelatin capsule, a liquid-filled hard gelatin capsule, a transdermal patch, a transdermal gel, a nasal drop, a nasal spray, a nasal powder, a nasal gel, a suppository, a rectal solution, a subcutaneous solution or emulsion, an intravenous solution or emulsion, an intramuscular injectable, intramuscular microspheres, intramuscular hydrogels, intramuscular organogels, intramuscular liquid crystals, an intramuscular solution, an inhalation solution, and an inhalation powder.

In one aspect, the minimum Cmax is 25 ng/mL. In one aspect, the minimum Cmax is 20 ng/mL. In one aspect, the minimum Cmax is 18 ng/mL.

In one aspect, the composition provides an extended release profile of the active agent of 1 week, 2 weeks, 3 weeks, or 4 weeks.

The preceding is a summary to provide an introduction and understanding of embodiments of the present disclosure. This summary is neither extensive nor exhaustive of the present disclosure and its various embodiments. The summary presents selected concepts of the embodiments of the present disclosure in a simplified form as an introduction to a more detailed description presented below. As will be appreciated, other embodiments of the present disclosure are possible utilizing, alone or in combination, one or more of the features set forth above or described in detail below.

The headings used herein are for organizational purposes and are not meant to be used to limit the scope of the description or the claims.

Any reference in the specification to “one embodiment” or “an embodiment” or “another embodiment” or a similar phrase means that a particular feature, structure, characteristic, operation, or function herein described is included in at least one embodiment. Thus, any appearance of the phrases “in one embodiment” or “in an embodiment” in the specification is not necessarily referring to the same embodiment. Further, the particular embodiments, aspects, features, structures, characteristics, operations, or functions may be combined in any suitable manner in one or more additional embodiments as if described verbatim, and it is intended that embodiments of the described subject matter can and do cover modifications and variations of the described embodiments. Particular aspects, as used herein, should be treated in a similar manner.

### BRIEF DESCRIPTION OF THE DRAWINGS

FIGS. 1 and 2 graphically present the Example 4, which is absolute bioavailability for an oral dose in the dog. The similarity of the curves between dogs, and between different days, demonstrates confidence that these measurements are predictive of the species. The same two dogs were each dosed 2042 IV at 10 mg/kg. The results are provided in Tables 4 and 5.

FIG. 3 graphically illustrates plasma NPT-2042 vs Time Plots (linear) for 10 mg/kg Bolus Dosing of NPT-2042 in Dogs (n=2).

FIG. 4 graphically illustrates plasma NPT-2042 vs Time Plots (log-linear) for 10 mg/kg Bolus Dosing of NPT-2042 in Dogs (n=2).

FIG. 5 graphically illustrates WinNonlin Plots for Lambda-z Estimation for 10 mg/kg Bolus Dosing of NPT-2042 in Dogs (n=2).

FIG. 6: Semi-log Plots of Mean Plasma Concentrations following IV (10 mg/kg) and Oral (30 mg/kg) Doses of NPT-2042.

FIG. 7: Linear and Semi-log Plots of Mean Plasma NPT-2042 Concentrations following IV (10 mg/kg) and Oral (30 mg/kg) Doses of NPT-2042.

### DETAILED DESCRIPTION OF THE INVENTION

The present disclosure relates to novel pharmaceutical compositions of bumetanide analogs, including bumetanide morpholinoamide, bumetanide diethylamide, and bumetanide dibenzylamide

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein may be used in practice or testing of the present disclosure. All publications, patent applications, patents and other references mentioned herein are incorporated by reference in their entirety. The materials, methods, and examples disclosed herein are illustrative only and not intended to be limiting.

As used herein, the articles “a,” “an,” and “the” are used herein to refer to one or to more than one (i.e., to at least one) of the grammatical object of the article. By way of example, “an element” can mean one element or more than one element.

As used herein, the term “about” or “approximately” refers to a quantity, level, value, number, frequency, percentage, dimension, size, amount, weight or length that varies by as much as 30, 25, 20, 15, 10, 9, 8, 7, 6, 5, 4, 3, 2 or 1 to a reference quantity, level, value, number, frequency, percentage, dimension, size, amount, weight or length. In some embodiments, the terms “about” or “approximately” when preceding a numerical value indicates the value plus or minus a range of 15%, 10%, 5%, or 1%.

As used herein, “an effective amount” refers to an amount that causes relief of symptoms of a disorder or disease as noted through clinical testing and evaluation, patient observation, and/or the like. An “effective amount” may further designate a dose that causes a detectable change in biological or chemical activity. The detectable changes may be detected and/or further quantified by one skilled in the art for the relevant mechanism or process. Moreover, an “effective amount” may designate an amount that maintains a desired physiological state, i.e., reduces or prevents significant decline and/or promotes improvement in the condition of interest. An “effective amount” may further refer to a therapeutically effective amount.

As used herein, the terms “individual” and “subject” are often used interchangeably and refer to any animal that may be treated with the methods disclosed herein. Suitable subjects (e.g., patients) include laboratory animals (such as mouse, rat, rabbit, or guinea pig), farm animals, and domestic animals or pets (such as a cat or dog). Non-human primates and human patients are included. In one embodiment, subjects may include human patients that have been diagnosed with a seizure disorder. As used herein, the term “patient” refers to a subject that may receive a treatment of a disease or condition.

As used herein, “treatment”, “treat”, and “treating” refer to reversing, alleviating, mitigating, or slowing the progression of, or inhibiting the progress of, a disorder or disease as described herein.

### Pharmaceutical Compositions

According to another aspect of the present disclosure, pharmaceutical compositions of bumetanide analogs as active ingredient, including bumetanide morpholinoamide, bumetanide diethylamide, and bumetanide dibenzylamide are described herein. In another aspect of the present disclosure is a pharmaceutical composition comprising an analog of bumetanide.

In some embodiments, the pharmaceutical compositions described herein may further include one or more additional therapeutic agents

The therapeutically effective dosage of the active ingredient of this disclosure may readily be determined for treatment of each desired indication. The amount of the active ingredient to be administered in the treatment of one of these conditions may vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.0001 mg/kg to about 10 mg/kg, and preferably from about 0.001 mg/kg to about 10 mg/kg body weight per day. A unit dosage may contain from about 0.05 mg to about 500 mg of active ingredient, and may be administered one or more times per day. The daily dosage for administration by injection, including intravenous, intramuscular, subcutaneous, and parenteral injections, and use of infusion techniques may be from about 0.0001 mg/kg to about 10 mg/kg. The daily rectal dosage regimen may be from 0.0001 mg/kg to 10 mg/kg of total body weight. The transdermal concentration may be that required to maintain a daily dose of from 0.0001 mg/kg to 10 mg/kg. The daily inhaled concentration may be that required to maintain a daily dose of from 0.0001 mg/kg to 10 mg/kg.

The specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present disclosure may be ascertained by those skilled in the art using conventional treatment tests.

The compounds of the present disclosure may be utilized to achieve the desired pharmacological effect by administration to a patient in need thereof in an appropriately formulated pharmaceutical composition. A patient, for the purpose of this disclosure, is a mammal, including a human, in need of treatment for a particular condition or disease. Therefore, the present disclosure includes pharmaceutical compositions which include a pharmaceutically acceptable carrier and a therapeutically effective amount of a compound as described herein. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A therapeutically effective amount of a compound is that amount which produces a result or exerts an influence on the particular condition being treated. The compounds described herein may be administered with a pharmaceutically-acceptable carrier using any effective conventional dosage unit forms, including, for example, immediate and timed release preparations, orally, parenterally, topically, nasally or the like. In some aspects, the compound is administered intravenously, orally, buccally, transdermally, or nasally. In yet another aspect, the compound is delivered so as to avoid or reduce the effects of first-pass metabolism.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which may be of the ordinary hard- or soft-shelled gelatin type containing, for example, surfactants, lubricants, and inert fillers such as lactose, sucrose, calcium phosphate, and corn starch.

In another aspect, the compounds of this disclosure may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings, or to otherwise modify, the physical form of the dosage unit. For instance, tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this disclosure may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soybean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds of this disclosure may also be administered parenterally, that is, subcutaneously, intravenously, intramuscularly, or intraperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as polyethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which may be used in the parenteral formulations of this disclosure are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quaternary ammonium salts, as well as mixtures.

The parenteral compositions of this disclosure may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant may be a single component having the above HLB or may be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

Another formulation employed in the methods of the present disclosure employs transdermal delivery devices (“patches”). Such transdermal patches may be used to provide continuous or discontinuous infusion of the compounds of the present disclosure in controlled amounts. The construction and use of transdermal patches for the delivery of pharmaceutical agents is well known in the art (See, e.g., U.S. Pat. No. 5,023,252, incorporated herein by reference). Such patches may be constructed for continuous, pulsatile, or on demand delivery of pharmaceutical agents.

Methods of delivering the compounds of this disclosure include any number of modes of administering the compounds or pharmaceutical compositions of compounds to the lungs via the nose or mouth, Modes of administration may include delivery of liquid or powder formulations of pharmaceutical compositions for nasal administration via either passive of active delivery mechanisms. Liquid formulations, may be delivered through a variety of mechanisms including vaporization through nasal inhalation, hand actuated nasal devices and mechanical spray pumps. Formulations for such delivery mechanisms may be in the form of propellant containing aerosols or propellant-free inhalable solutions. Mechanical spray pumps may be hand actuated, gas driven or electrical, as in the case of electrically powered nebulizers and atomizers. In one aspect described herein, a propellant-free inhalable solution is administered by nebulizer or direct nasal inhalation.

Powder formulations may be delivered though mechanical power sprayers, nasal inhalers and nebulizers/atomizers. Prior to delivery, powder formulations may be solubilized in suitable solvents including water and saline solutions. In one aspect described herein the compound may be solubilized in a saline solution. In another aspect described herein a therapeutically effective amount of the compound delivered to the lungs.

The compositions of the disclosure may also contain other conventional pharmaceutically acceptable compounding ingredients, generally referred to as carriers or diluents, as necessary or desired. Any of the compositions of this disclosure may be preserved by the addition of an antioxidant such as ascorbic acid or by other suitable preservatives. Conventional procedures for preparing such compositions in appropriate dosage forms may be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, or trolamine.

Other pharmaceutical ingredients include, for example, but are not limited to, adsorbents (e.g., powdered cellulose and activated charcoal); aerosol propellants (e.g., carbon dioxide, CCl2F2, F2ClC-CClF2 and CClF3); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (includes but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable on); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

Methods/Uses

The compounds of the present disclosure are useful in the treatment of a variety of seizure disorders, otherwise known as epilepsies and epileptic syndromes. Reference is made to The International Classification of Epilepsies and Epileptic Syndromes [adapted from Commission on Classification and Terminology of the International League Against Epilepsy. Proposal or revised classification of epilepsies and epileptic syndromes. Epilepsia 1989; 30:389-99.]: https://www.epilepsy.com/learn/professionals/about-epilepsy-seizures/overview-epilepsy-syndromes, herein incorporated by reference with regard to such diseases and disorders, as well as their diagnoses.

Localization-related (focal, local, partial) epilepsies and syndromes
a. Idiopathic with age-related onset
   i. Benign childhood epilepsy with centrotemporal spikes
   ii. Childhood epilepsy with occipital paroxysms
b. Symptomatic
   i. Chronic progressive epilepsia partialis continua of childhood
   ii. Syndromes characterized by seizures with specific modes of precipitation
   iii. Temporal lobe epilepsies
   iv. Frontal lobe epilepsies
   v. Parietal lobe epilepsies
   vi. Occipital lobe epilepsies
c. Crytopgenic

Generalized epilepsies and syndromes
a. Idiopathic, with age-related onset (listed in order of age)
   i. Benign neonatal familial convulsions
   ii. Benign neonatal convulsions
   iii. Benign myoclonic epilepsy in infancy
   iv. Childhood absence epilepsy (pyknolepsy)
   v. Juvenile absence epilepsy
   vi. Juvenile myoclonic epilepsy (impulsive petit mal)
   vii. Epilepsy with grand mal seizures on awakening
   viii. Other generalized idiopathic epilepsies not defined above
   ix. Epilepsies with seizures precipitated by specific modes of activation
b. Idiopathic and/or symptomatic (listed in order of age)
   i. West syndrome (infantile spasms)
   ii. Lennox-Gastaut syndrome
   iii. Epilepsy with myoclonic-astatic seizures
   iv. Epilepsy with myoclonic absences
c. Symptomatic
   i. Nonspecific etiology
      1. Early myoclonic encephalopathy
      2. Early infantile epileptic encephalopathy with suppression burst
      3. Other symptomatic generalized epilepsies not defined above
   ii. Specific etiology
      1. Epileptic seizures may complicate many disease states

Epilepsies and syndromes undetermined as to whether they are focal or generalized
a. With both generalized and focal seizures
   i. Neonatal seizures
   ii. Severe myoclonic epilepsy in infancy
   iii. Epilepsy with continuous spike waves during slow-wave sleep
   iv. Acquired epileptic aphasia (Landau-Kleffner syndrome)
   v. Other undetermined epilepsies not defined above
b. Without unequivocal generalized or focal features

Special syndromes
a. Situation-related seizures
   i. Febrile convulsions
   ii. Isolated, apparently unprovoked epileptic events
   iii. Seizures related to other identifiable situations such as stress, hormonal changes, drugs, alcohol, or sleep deprivation

Incidence and Risk Factors

According to the World Health Organization, epilepsy accounts for 1% of the global burden of disease [1]. Epilepsy affects 1%-3%-of the U.S. population [19]. Factors that increase the risk of developing epileptic seizures by more than ten-fold include cerebral palsy, mental retardation, febrile seizures, clinically detected stroke, Alzheimer disease, infection of the central nervous system (CNS) and head trauma [2]. For example, a study of Vietnam veterans who survived penetrating head wounds showed that the risk for developing seizures was increased 580-fold during the first year after the injury, and 25-fold after ten years [2,3]. Although there are currently no recommended treatments or approved therapeutics for reducing the risk of developing epilepsy, a population-based control study in the elderly showed that the loop diuretic furosemide (Lasix) was protective for the development of epilepsy, with a protective factor of 39% [4] (and hence, so too may be bumetanide).

Medically Intractable Epilepsy

Seizures that cannot be controlled by existing pharmacotherapeutics are referred to in a number of different ways, including “uncontrolled,” “intractable,” “refractory,” “drug resistant”, or “medically resistant”. It is estimated that 20%-40% of patients with epilepsy (approximately 400,000 Americans) have refractory epilepsy [5]. The total indirect and direct cost of epilepsy in the United States is estimated to be $15.5 billion yearly, with drug resistant patients contributing a major proportion of this cost [6], [7]. In spite of the availability of numerous new drugs to treat epilepsy over the last decade, the efficacy of these new drugs have not proven to be significantly better than older drugs [8].

Most common AEDs (and their use to treat other neurological and psychiatric disorders in addition to epilepsy).

The most commonly prescribed AEDs include valproate (VPA—the most commonly prescribed of all AEDs worldwide.) and its derivative Divalproex Sodium, carbamazepine (Tegretol), phenytoin (Dilantin), the barbiturates (Phenobarbitol and Primidone), ethosuximide (Zarontin), clonazepam (Klonopin), lamotrigine (Lamictal), gabapentin (Neurontin), topiramate (Topamax), oxcarbazeipin (Trileptal), and Zonisamide (Zonegran) [11]. In addition to their use for treating epilepsy, AEDs are also prescribed to treat numerous other neurological and psychiatric disorders [10].

Side Effects

All currently prescribed antiepileptic drugs (AEDs) are thought to mediate their antiepileptic effects by reducing neuronal or synaptic excitability. Since AEDs affect all neuronal or synaptic targets in the brain indiscriminately, regardless of whether or not they contribute to seizure activity, all AEDs also mediate a spectrum of cognitive, neurological, and psychiatric sided effects[18]. Approximately 25% of patients discontinue their treatment because of intolerable side effects [12,13]. Treatment failure and poor adherence are very common in patients who experience side-effects from their AEDs. The negative consequences of side-effects can significantly affect the lives of relatives and friends of the patient. Commonly occurring side effects of AEDs include memory problems, fatigue, tremors, gastrointestinal symptoms, osteoporosis, depression, drowsiness, weight gain, nausea, and numerous others [14]. One study in The Netherlands estimated the economic costs of the side effects of epilepsy (in addition to the direct and indirect costs of epilepsy itself) for patients in that country to be $26,675 USD per patient per year. (To understanding, this is the only study to date that attempted to quantify the economic burden of the side effects of AEDs).

All commonly used AEDs have some effect on cognition, and these effects can have a considerable impact on epilepsy patients when crucial functions are involved, such as learning in children [17]. The most prevalent of the CNS adverse effects on cognition of CNS drugs are sedation, somnolence, distractibility, insomnia, and dizziness [20].

Fatigue is a common side effect of most antiepileptic drugs [15]. Fatigue induced by AEDs is a chronic condition that can negatively affect the patients work, social interactions, and family. Stimulants such as amphetamine, dextroamphetamine, and methylphenidate are sometimes used to treat fatigue and daytime somnolence. However, these medications can increase seizure intensity and lower seizure threshold, and hence it isn't desirable to use these drugs to treat fatigue in epilepsy patients [16].

All antiepileptic drugs are thought to increase the risk of suicidal thoughts or actions. This risk is of sufficient concern that the FDA issued safety alerts on Dec. 15, 2008 and Jan. 31, 2008, and currently requires the labeling of all AEDs to include a warning about an increased risk of suicidal thoughts or actions [9]. This is particularly problematic since epilepsy and other illnesses and psychiatric conditions for which AEDs are prescribed as a treatment (chronic pain, depression, bipolar disorders, and anxiety) all inherently have an increased risk of suicidal behavior [10]. For example, death by suicide in people with epilepsy is more common than the population as a whole (5% vs. 1.4%) [50]. Thus, the use of an AED to treat a disorder that already has an associated risk of suicidal behavior, would be expected to further increase that risk.

Every antiepileptic drug studied to date has been shown to have endocrine side effects in both men and women [21]. These can adversely affect fertility, sexuality, thyroid function, bone health. AEDs can alter levels of sex hormones, which can cause menstrual disturbances, sexual problems, and reduced fertility. Other side effects that affect physical appearance include weight gain, alopecia (hair loss), acne, and masculine hair distribution in women [22].

Poor Patient Adherence to Existing Antiepileptic Drugs

As stated above, approximately 25% of patients discontinue their treatment because of intolerable side effects [12,13]. Up to 50% of all epilepsy patients develop adverse reactions to AEDs, which in turn negatively effects tolerability and adherence [23]. Even if a side effect is not intolerable, if it is unpleasant it can reduce patient adherence to taking their AEDs as prescribed. Non-adherence in epilepsy is estimated to be in the range from 30%-50% [24, 25]. Decreased AED adherence is associated with more than a 3-fold increase in mortality [26]. Periods of nonadherence in patients with epilepsy were also associated with significantly more emergency department visits, hospital admissions, injuries, and fractures [25].

Comorbidities

General: A recent study determined the prevalence of the most common comorbidities in men and women with epilepsy based on data from commercial health plans [46]. The top 10 comorbidities for women and their relative prevalences were psychiatric diagnosis (16%), hypertension (12%), asthma (11%), hyperlipidemia (11%), headache (7%), diabetes (6%), urinary tract infection (5%), hypothyroidism (5%), anemia (5%), and migraine (4%). For men, the top 10 comorbidities and their relative prevalences were psychiatric diagnosis (15%), hyperlipidemia (12%), hypertension (12%), asthma (8%), diabetes (5%), headache (4%), cancer (4%), coronary artery disease (3%), anemia (3%), and gastroesophageal reflux disease (3%). Seven of the top 10 comorbidities were common to both women and men. Psychiatric diagnosis was the only comorbidity among the top five comorbidities for all age groups. The presence of one comorbidity approximately tripled the health-care cost for that member compared with the cost for members who had no comorbidities.

Psychiatric disorders—general: Epilepsy increases the likelihood of depression, anxiety disorders, attention deficit hyperactivity disorder (ADHD), schizophrenia-like interictal psychosis, autism, as well as suicidal behavior. Likewise, individuals with these psychiatric diagnoses and suicidal behavior are more likely to have epilepsy [27-35].

Sleep disorders: Sleep deprivation is known to lower seizure thresholds in people with epilepsy [36]. Sleep is vulnerable to its own set of disorders that can disrupt it. One example is obstructive sleep apnea [OSA]. Both adults and children with refractory epilepsy are at much higher risk than the normal population for developing OSA [37].

Autism Spectrum Disorder (ASD): Epilepsy occurs at a much higher frequency in individuals with autism. Between 11% and 39% of individuals with autism develop epilepsy [38]. Epilepsy and autism co-exist in up to 20% of children with either disorder. In children with autism, the highest prevalence of epilepsy is in those with intellectual disability [39].

Sleep disturbances in children with autism are prevalent, with estimates from 40% to 80% of children being affected [36].

The atypical antipsychotic medications risperidone and aripiprazole are approved by the food and drug administration for the treatment of irritability and agitation in ASD. Both are associated with significant adverse events, including the lowering the seizure threshold [40].

Migraine: Migraine has an incidence of around 1% per year and a 1-year prevalence of 11.7-13.2% Patients with epilepsy have a roughly twofold increased risk of having migraine as well [42]. Conversely, children with migraine have threefold to fourfold increase in the risk of developing epilepsy.

Comorbidity can worsen outcome. Patients with epilepsy who suffer from migraine are less likely to have a remission of epilepsy than those with epilepsy alone [43]. This is also evidence for a complex comorbid cluster of epilepsy, migraine, depression, and suicide [44, 45].

Postictal headaches—45% of people with epilepsy have headaches following seizures, called postical headache. These headaches last between 6-24 hours or longer, and can be quite disabling[64]. A number of drugs used to treat these headaches can lower seizure threshold, increasing risk for further seizures.[16]

Depression: Depressive disorders in patients with epilepsy has been shown to range between 9% and 55% depending on the sample population and the methods of assessment [47]. This is in contrast to the prevalence in the general population estimated to be 1%-3% in men, and 2%-9% in women [48].

The therapeutics commonly used to treat depression can lower seizure thresholds or increase the severity of seizures. Bupropion and tricyclic antidepressants reduce seizure threshold [16]. Selective serotonin reuptake inhibitors (SSRIs) can significantly prolong seizures [49].

Anxiety: The lifetime prevalence of anxiety is estimated to be 2.4 times higher in people with epilepsy than in people without epilepsy [51].

Psychosis: The risk of psychosis in patients with epilepsy may be 6-12 times that of the general population, with a prevalence of about 7-8% [52]. All antipsychotic medications can lower seizure thresholds. [16].

Attention Deficit Disorder (ADD) and Attention Deficit/Hyperactivity Disorder (ADHD):

Nearly 20% of adults diagnosed with epilepsy also show symptoms of ADHD [53]. Studies in pediatric epilepsy have found a 2.5-fold to 5.5-fold-increased risk of ADHD compared with healthy controls[54-58]. It is estimated that between 2% and 7% of children with ADHD have epilepsy [59-61].

Stimulants such as amphetamine, dextroamphetamine, and methylphenidate are commonly prescribed to treat Attention Deficit Disorder (ADD) and Attention Deficit/Hyperactivity Disorder (ADHD) in children as well as adults [16]. These drugs can reduce seizure threshold and increase seizure severity.

Mental retardation: Epilepsy is one of the most common secondary disabilities in people with mental retardation, the prevalence increasing with the severity of the intellectual disability. About 50% of those with profound learning disability develop epilepsy. The prevalence of lifetime epilepsy among people with mental retardation (IQ<70) is between 13% and 24% [66]. Down Syndrome is the most common genetic cause of mental retardation; the number of people with Down Syndrome who have seizures is estimated to be between 5% and 10% [67]. Currently available AEDs and elicit adverse behavioral effects in individuals with mental retardation [68].

Others: Hyperlipidemia—incidence rate is 1.3 fold higher in epilepsy patients than in control [62]. Population-based surveys document higher rates of hypertension, ischemic heart disease and diabetes in people with epilepsy [63].

Drug Interactions:

All of the following drugs can lower seizure threshold, thus increasing the risk of seizures in people with epilepsy [16]:
Acetylcholinesterase inhibitors—Used to treat: Myasthenia gravis, glaucoma, postural tachycardia syndrome, neuropsychiatric symptoms of Alzheimer's disease, Lewy Body Dementia, Parkinson's disease, cognitive impairments in schizophrenia, autism.
Anticholinergics—Used to treat gastrointestinal disorders, genitourinary disorders, respiratory disorders, sinus bradycardia, insomnia, and dizziness.
Antiemetics—Used to treat nausea/vomiting.
Antihistamines—suppress symptoms of allergic reaction.
Baclofen—skeletal muscle relaxant for spasticity.
β-Blockers—Angina pectoris, Atrial fibrillation, Cardiac arrhythmia, Congestive heart failure, Essential tremor, Glaucoma, Hypertension, Migraine prophylaxis, Mitral valve, prolapsed, Myocardial infarction, Phaeochromocytoma, in conjunction with α-blocker, Postural orthostatic tachycardia syndrome, Symptomatic control (tachycardia, tremor) in anxiety and hyperthyroidism, Theophylline overdose
Cephalosporins—antibiotics
Cyclosporine—immunosuppressant, used for severe rheumatoid arthritis, severe psoriasis,
Dalfampridine—(nasty freaking stuff—I used it in the lab to induce severe seizures)—used to treat multiple sclerosis, spinal cord injury, Parkinson's disease.
Estrogen—oral contraceptives, hormonal replacement therapy (given postmenopause to prevent osteoporosis, treat the symptoms of menopause, prostate cancer,
Imipenem—antibiotic
Iodinated Contrast Dyes—radiocontrast agent.
Isoniazid—prevention and treatment of tuberculosis.
Lithium—bipolar disorders, major depression, and schizophrenia.
Local anesthetics—seizures are a well recognized side effect due to the administration of local anesthetics.
Methotrexate—chemotherapy for certain cancers, autoimmune disorders including rheumatoid arthritis, juvenile dermatomyositis, psoriasis, psoriatic arthritis, lupus, sarcoidosis, Crohn's disease, eczema and many forms of vasculitis.
Metronidazole—antibiotic and antiprotozoal medication.
Narcotics—pain
Penicillins—antibiotics
Pyrimethamine—antimalarial drug, used for protozoal infections.
Quinolones—antibacterial drugs.
Theophylline—chronic obstructive pulmonary disease (COPD), asthma, infant apnea. Blocks the action of adenosine, an inhibitor neurotransmitter that induces sleep, contracts the smooth muscles and relaxes the cardiac muscle.
Tramadol—pain.

Rarer Epilepsy Syndromes [65]
Angelman Syndrome—Occurs in 1/15,000 births. Epilepsy is present in more than 80% of affected individuals.
Benign Rolandic Epilepsy—Represents about 15% of all epilepsies in children. Seizures stop by age 15.
CDKLS Disorder—Quite rare, 600 cases worldwide.
Childhood Absence Epilepsy—Accounts for 2-8% of people with epilepsy. Usually disappears by adulthood.
Dravet Syndrome: Affects 1/30,000. Myclonic seizures appear between 1-5 years in 85% of children.
GLUT1 deficiency syndrome—Maybe 1/90,000, but thought to be under-diagnosed because many neurological disorders cause similar symptoms. Almost all individuals have frequent seizures beginning in first year of life.
Hypothalamic hamartom—1/200,000.
Infantile spasms (also known as West syndrome)—2.5-6 of every 10,000 births. Accounts for 30% of all cases of epilepsy affecting infants. Usually stops by 4 yrs, but most children left developmentally impaired, and one-fifth will have Lennox-Gastaut syndrome. Many clinicians believe that the sooner the seizures are controlled, but better the outcome.
Lennox-Gastaut—accounts for 2-5% of childhood epilepsies. Usually persists through childhood and adolescence to adult years. Seizures very hard to control with current therapeutics.
PCDH19—1 in 10 girls that begin giving seizures before age 5 may have PCDH19 epilepsy. Can overlap or look similar to Dravet Syndrome. 15,000-30,000 people in with PCEH19 epilepsy in the United States.
Progressive myoclonic epilepsy—Not a single disorder, but includes a group of syndromes with various names, including “Severe myoclonic epilepsy of infancy (Dravet syndrome), Unverrict-Lundbord disease (also called Baltic myoclonus), Lafora disease, and Mitochondrial encephalopathies. Hard to control any of these patients with existing therapteutics.
Rasmussen's Encephalitis—Nothing is known about its incidence in different populations. Considered rare, but clinicians all over the world describe patients with this syndrome. Outlook grim with current therapeutics; seizures are relentless.
Ring Chromosome 20 Syndrome—rare.
Reflex epilepsies—Group of epilepsy syndromes in which a certain stimulus (e.g. flickering light) triggers seizures. 4-7% among patients with epilepsy.

Monontherapy vs polytherapy (combination therapy) side effects. Often a single AED might provide partial, but inadequate seizure control. For patients who are refractory to any single AED (monotherapy), better seizure control is sometimes obtained by combining several different AEDs (polytherapy, or combination therapy). Sometimes up to four AEDs are given to a single patient to try to control seizures. However, the intensity and number of side effects significantly increases when more than one AED is given to a patient [69]. A recent study has showed that polytherapy has more severe and numerous cognitive side effects. The intensity and number of side effects increases with the number of AEDs a patient is taking [70].

Loop Diuretics Furosemide and Bumetanide:

Furosemide and bumetanide are “loop diuretics” (diuretics that act at the ascending loop of Henle in the kidney) that are primarily used to treat hypertension and edema. These drugs mediate their diuretic effects by antagonizing the NKCC2 cotransporter in the kidney. Bumetanide is 40-100× more potently diuretic than furosemide, and is often used in patients for whom high doses of furosemide are not adequately effective.

Furosemide and bumetanide have been safely used as diuretics for decades in millions of patients. These loop diuretics have never been reported to elicit neurological, psychiatric, or cognitive side effects.

The loop diuretics have also been shown to mediate certain therapeutic effects in the central nervous system (CNS), presumably through its effects on the neuronal and glial isoform for the NKCC cotransporter, called NKCC1. For example, bumetanide and furosemide have been shown to significantly reduce seizures in animal epilepsy models and patients suffering from refractory epilepsy [71-74]. Furosemide and bumetanide have also been shown to elicit anxiolytic effects in animal models of anxiety [75]. Bumetanide has been shown to prevent alterations in sociability and functional brain connectivity cause by early-life seizures, and may then prevent the autistic-like behaviors that may result from early-life seizures [76]. Bumetanide has also been shown to improve behavioral symptoms in children with autism or Asperger syndrome [77]. The loop diuretics have been shown to have potential therapeutic effects in preventing and treating migraine [78-80]. The loop diuretics have also been shown to have potential therapeutic effects for treating inner ear disorders, including tinnitus [81], neuropathic pain [82], and for rescuing cognitive disabilities in individuals with Down Syndrome [83].

As mentioned, furosemide and bumetanide are diuretics, with bumetanide being 40-100× more potently diuretic than furosemide. Bumtanide is also a much more potent and specific antagonist than is furosemide for NKCC1 in brain cells, and hence may be expected to elicit its CNS therapeutic effects more potently than furosemide. These loop diuretics were designed for the purpose of acting systemically to induce diuresis via their action on NKCC2 in the kidney, and are thought to have poor blood-brain-barrier penetration. Large doses of these diuretics may be required to mediate their therapeutic effects when treating CNS diseases in humans. Increased frequency of urination caused by these drugs is an unpleasant side effect. Increased urination frequency could disturb sleep, which is problematic for patients suffering from CNS disorders (described above).

The FDA labeling for bumetanide includes the statement that serum potassium should be measured periodically and potassium supplements or potassium sparing diuretics added if necessary. Periodic determinations of other electrolytes are advised in patients treated with high doses or for prolonged periods, particularly in those on low-salt diets. Hyperuricemia may occur; it has been asymptomatic in cases reported to date. Reversible elevations of the BUN and creatinine may also occur, especially in association with dehydration and particularly in patients with renal insufficiency. Bumex may increase urinary calcium excretion with resultant hypocalcemia. Diuretics have been shown to increase the urinary excretion of magnesium; this may result in hypomagnesemia.

Ideally, it would be desirable to increase the ability of the loop diuretics to pass the blood-brain-barrier. This would have the effect of being able to reach greater therapeutic effects in the brain, and reduced diuretic effects. We have found that certain amide analogs and prodrugs of bumetanide have profound antiepileptic effects, with dramatically reduced diuretic effects by comparison to bumetanide (see figures). This is an unexpected discovery. For example, Tollner et al [84] tested a bumetanide amide prodrug in their studies (N,N-Dimethylaminoethylamide) in rats, and found that it did not lead to enhanced levels of bumetanide. The authors in this study then chose to abandon further testing of amide prodrugs of bumetanide.

Compounds Used in the Present Invention

In one embodiment of the present disclosure, methods to treat epilepsy patients who are not well-controlled by or otherwise refractory to conventional therapies, such as phenytoin, carbamazepine, valproate, lamotrigine, levetiracetam, ethosuximide, phenobarbital, and topiramate by administering to the patient a pharmaceutical composition containing a compound of the present disclosure. Also provided is a method of treating epilepsy with a derivative of a known loop diuretic without need for a potassium supplement. The method of the invention provides a method of addressing the risk of epilepsy in patients with an associated condition, such as cerebral palsy, brain injury, stroke, brain tumor, substance use disorders or genetic mutations, which are linked to a small proportion of the disease. Another aspect of the invention is a unit dose of a compound of the preset disclosure for oral administration containing molar amount or weight than the equipotent amount of the parent acid in an anticonvulsant model.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

### SYNTHETIC EXAMPLES

### Example 1: N-Morpholinyl 3-Aminosulfonyl-5-N-butylamino-4-phenoxybenzamide (Bumetanide N-Morpholinoamide (may be referred to as NPT-2043)

The reaction was conducted using bumetanide (11.48 g, 31.5 mmol), HOBt (2.13 g, 15.8 mmol), morpholine (13.72 g, 158 mmol), and EDC (9.66 g, 50.4 mmol) in THF (160 mL). After 5 hours, the reaction was 85% completed, so additional EDC (2 g, 10.4 mmol) was added, and the reaction was heated to 50° C. The reaction mixture was cooled, and the solvent was evaporated. The residue was partitioned between dichloromethane (150 mL), water (100 mL), and 1N sodium hydroxide (20 mL). The organic phase was then washed with water (100 mL), dried over anhydrous sodium sulfate, filtered, evaporated, and the residue was further purified by silica gel chromatography (25% ethyl acetate in dichloromethane) to yield 12.63 g (92%) of N-morpholinyl 3-aminosulfonyl-5-N-butylamino-4-phenoxybenzamide.

### Example 2: N,N-Diethyl 3-Aminosulfonyl-5-butylamino-4-phenoxybenzamide (Bumetanide Diethylamide)

Bumetanide (1.16 g, 3.2 mmol) was dissolved in dichloromethane (10 mL) and 1-ethyl-3-(3-dimethylaminopropyl)carbodiimide (EDC, 690 mg, 3.6 mmol) was added. After 5 minutes N-hydroxybenzotriazole (HOBt, 498 mg, 3.6 mmol) was added and the solution was stirred for an additional 5 minutes. Diethylamine (332 uL, 3.2 mmol) was added and the reaction was stirred for 2 hours. The reaction was washed with saturated sodium bicarbonate, water and brine, and dried with magnesium sulfate. The dichloromethane was removed under reduced pressure to yield 860 mg (65%) of pure N,N-diethyl 3-aminosulfonyl-5-butylamino-4-phenoxybenzamide.

### Example 3: N,N-Dibenzyl 3-Aminosulfonyl-5-butylamino-4-phenoxybenzamide (Bumetanide Dibenzylamide), (“NPT2042”)

Bumetanide (960 mg, 2.6 mmol) was dissolved in dimethylformamide (DMF, 10 mL) and 1-ethyl-3-(3-dimethylaminopropyl)carbodiimide (EDC, 560 mg, 3.6 mmol) was added. After 10 minutes 1-hydroxybenzotriazole (HOBt, 392 mg, 2.9 mmol) was added and the solution was stirred for an additional 10 minutes. Dibenzylamine (1 mL, 5.2 mmol) was added and the reaction was stirred for 2 hours, at which time the reaction was complete by LC/MS, The reaction was poured into saturated ammonium chloride (20 mL) and extracted with ethyl acetate (2×100 mL). The ethyl acetate, was washed with saturated sodium bicarbonate, water and brine, and dried over anhydrous magnesium sulfate. The ethyl acetate was removed under reduced pressure to yield 1.0 g (75%) of N,N-dibenzyl 3-aminosulfonyl-5-butylamino-4-phenoxybenzamide as white solid.

U.S. Pat. No. 8,008,283 is hereby incorporated by reference in its entirety, US 2017/0246131 is herein incorporated by reference in its entirety,

### Formulation Examples

In certain embodiments of the present disclosure, the active agent is provided in a composition is selected from the group consisting of: a sublingual tablet, a sublingual amorphous solid dispersion, an orodispersible film, a fast dissolving film, a nanoparticle spray, a muco-adhesion patch, a liquid, a slick pack, a lipid-filled soft gelatin capsule, a liquid-filled hard gelatin capsule, a transdermal patch, a transdermal gel, a nasal drop, a nasal spray, a nasal powder, a nasal gel, a suppository, a rectal solution, a subcutaneous solution or emulsion, an intravenous solution or emulsion, an intramuscular injectable, intramuscular microspheres, intramuscular hydrogels, intramuscular organogels, intramuscular liquid crystals, an intramuscular solution, an inhalation solution, and an inhalation powder.

In one embodiment, the pharmaceutical composition is for oral administration.

NPT2042 may be formulated in an “extended release” or “long lasting” formulation. This is of benefit for conditions where patients have poor adherence. It is especially important for epilepsy because SUDEP (Sudden Unexpected Death in Epilepsy) occurs most frequently in epilepsy patients who forget to take their last dose of drug.

The present disclosure includes extended release formulations that can last over different time durations.

Regarding a nasal (intranasal) administration, the formulation can target either the intranasal mucosa to deliver an active agent systemically to the blood while bypassing first-pass metabolism, or it can transport drugs directly to the brain from the nasal cavity along the olfactory and trigeminal nerves. In one embodiment, the direct administration to the olfactory and trigeminal nerves may be preferred. In one embodiment, reference is made to https://www.intechopen.com/books/drug-discovery-and-development-from-molecules-to-medicine/intranasal-drug-administration-an-attractive-delivery-route-for-some-drugs, incorporated by reference with regard to such teaching. Another embodiment to address the direct access to brain route: https://pubmed.ncbi.nlm.nih.gov/29277310/ is incorporated by reference with regard to such teaching.

One embodiment of the present disclosure includes “PO Lympatic-targeted” formulations. NPT2042 is extremely lipophilic. Some such drugs may be formulated with lipids/oils or various other technologies so that when swallowed, they are absorbed by the lymphatic system in GI tract, and thus avoid first-pass metabolism.

### Prophetic Examples of Formulations

TABLE 1

| | | | |
|---|---|---|---|
| | | Extended | |
| Route | Immediate Release | Release* | Reference |

| | | |
|---|---|---|
| Sublingual | Tablet | |
| Sublingual | Thin Film | |
| Sublingual | Liquid with blow-fill seal | |
| Sublingual | Liquid with dropper | |
| Sublingual | Stick pack | |
| PO Lymphatic- | Soft Gelatin Capsule | |
| targeted | | |
| PO Lymphatic- | Liquid Filled Hard | |
| targeted | Gelatin Capsule | |
| Transdermal | | Depot |
| Transdermal | Patch | Patch |
| Transdermal | Thin film | |
| Nasal | Sterile Solution | |
| Rectal | Suppository | |
| Rectal | Solution | |
| Subcutaneous | Microneedles | |
| Subcutaneous | SC Solution | |
| Intravenous | IV Sterile solution | |
| Intramuscular | IM Solution | IM Solution |
| Inhalation | Sterile Solution | |
| Inhalation | Powder | |

*All Extended Release: 1 week, 2 weeks, 3 weeks, 4 weeks

TABLE 2

| Route | Presentation |
|---|---|
| Sublingual (IR) | Tablet |
| | Tablet, amorphous solid dispersion |
| | Orodispersible Film |
| | Fast dissolving Film |
| | Spray, nanoparticle |
| | Muco-adhesion |
| | Liquid with blow-fill seal |
| | Stick pack |
| PO Lymphatic- | Liquid Filled Soft Gelatin Capsule |
| targeted (IR) | Liquid Filled Hard Gelatin Capsule |
| Transdermal (ER) | Patch |
| Transdermal (IR) | Gel |
| Nasal (IR) | Drops |
| | Sprays |
| | Powder |
| | Gels |
| Rectal (IR) | Suppository |
| | Solution |
| Subcutaneous (IR) | Microneedles |
| | Solution |
| Intravenous (IR) | Solution |
| Intramuscular (ER) | Long-acting Injectable, microspheres, |
| | hydrogels, organogels, and liquid crystals |
| Intramuscular (IR) | IM Solution |
| Inhalation (IR) | Solution |
| | Powder |

Reference is made to one or more of Narang, 2011, Ibrahim, 2018, Hoffmann 2011, Morales, 2011, Baltzley 2018, Duggan 2017, O'Driscoll, 2002, O'Driscoll, 2002, Jadhav, 2007

### Pharmacokinetic Examples

Assays were performed to determine absolute bioavailability of NPT2042 in two species: dog and rat. The calculation of absolute bioavailability depends on four inputs: the areas under the curves (AUC) for oral and IV, and the oral and IV doses.

### Example 4: Dog

Testing was conducted to determine AUCs for an oral dose in dog.

Two dogs were each dosed 30 mg/kg NPT2042 orally, on two different days. The results are provided in Table 3.

TABLE 3

| | Dose Level | | Cmax | Tmax | AUClast | AUCinf | t½ | CL/F | Vz/F |
|---|---|---|---|---|---|---|---|---|---|
| Route | (mg/kg) | Animal | (ng/mL) | (h) | (h * ng/mL) | (h * ng/mL) | (h) | (L/h/kg) | (L/kg) |
| Oral | 30 | D0001 | 151 | 6.00 | 1340 | 1360 | 3.61 | 22.0 | 115 |
| Oral | 30 | D0601 | 153 | 6.00 | 1450 | 1520 | 4.72 | 19.7 | 134 |
| | | Mean | 152 | 6.00 | 1390 | 1440 | 4.16 | 20.8 | 124 |
| | | Min | 151 | 6.00 | 1340 | 1360 | 3.61 | 19.7 | 115 |
| | | Max | 153 | 6.00 | 1450 | 1520 | 4.72 | 22.0 | 134 |

Based on Example 4, absolute bioavailability for an oral dose in the dog was 5.1%.

Absolute bioavailability was determined as (AUCpo/AUCiv)*(Doseiv/Dosepo)=(1440/9430)*(10/30)=0.0509=5.1%

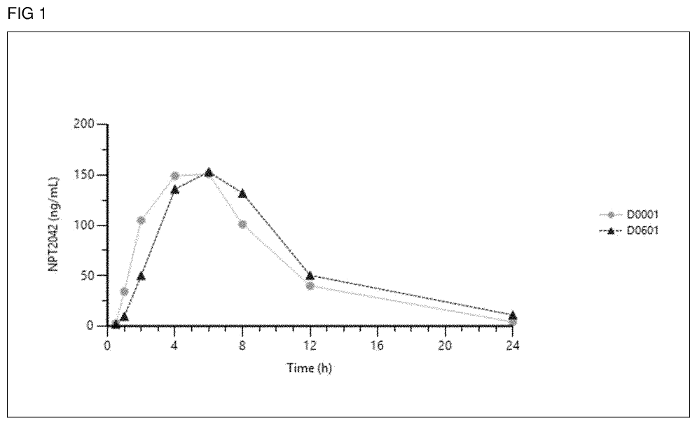

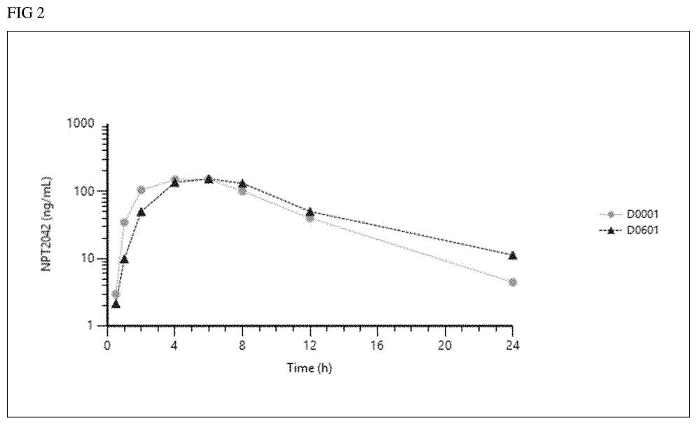

The data is graphically presented on FIG. 1 and FIG. 2. The similarity of the curves between dogs, and between different days, demonstrates confidence that these measurements are predictive of the species.

Testing was conducted to determine AUC for an IV dose in dog.

The same two dogs were each dosed 2042 IV at 10 mg/kg. The results are provided in Tables 4 and 5.

TABLE 4

| | | Time | | | | | | | |
|---|---|---|---|---|---|---|---|---|---|
| | | (h) | | | | | | | |
| Dose | | 0.50 | 1.00 | 2.00 | 4.00 | 6.00 | 8.00 | 12.00 | 24.00 |
| Level | | Bumetanide | | | | | | | |
| (mg/kg) | Animal | (ng/ml) | | | | | | | |
| 30 | D0001 | BQL | BQL | BQL | BQL | BQL | BQL | BQL | BQL |
| | D0601 | BQL | BQL | BQL | BQL | BQL | BQL | BQL | BQL |
| | Mean | | | | | | | | |
| | Min | | | | | | | | |
| | Max | | | | | | | | |

TABLE 5

| | | Time | | | | | | | |
|---|---|---|---|---|---|---|---|---|---|
| | | (h) | | | | | | | |
| Dose | | 0.50 | 1.00 | 2.00 | 4.00 | 6.00 | 8.00 | 12.00 | 24.00 |
| Level | | Bumetanide | | | | | | | |
| (mg/kg) | Animal | (ng/ml) | | | | | | | |
| 30 | D0001 | BQL | BQL | BQL | BQL | BQL | BQL | BQL | BQL |
| | D0601 | BQL | BQL | BQL | BQL | BQL | BQL | BQL | BQL |
| | Mean | | | | | | | | |
| | Min | | | | | | | | |
| | Max | | | | | | | | |

Plasma NPT-2042 Concentrations were high and still quantifiable at 24 hours after dosing.

Bumetanide concentrations were only measurable at 5 min (both dogs) and 1 hour (1 dog) indicating minimal metabolism of NPT-2042 to bumetanide and likely explaining the lack of diuresis.

Concentration-time profile for both dogs were very similar.

NPT-2042 displayed multi-exponential decay after bolus dosing (see log-linear plots)

The concentration at 5 minutes after dosing averaged 5560 ng/mL and the back-extrapolated concentration (CO) averaged 6060 ng/mL.

The terminal-phase half-life (see WinNonlin plots) averaged 4.25 hours.

The total body clearance (CL) averaged 1.07 L/h/kg. Hepatic blood flow averages about 1.5 L/h/kg in dogs and so the CL represents about ⅔ of hepatic blood flow indicating the NPT-2042 is a relatively high clearance drug in dogs.

The volume of distribution (Vz) averaged 6.49 L/kg and was very consistent between the two animals. NPT-2042 has a relatively large volume of distribution which is not surprising based on its lipophilicity.

TABLE 6
Plasma Concentration-Time Data

| | | Time | | | | | | |
|---|---|---|---|---|---|---|---|---|
| | | (h) | | | | | | |
| | | 0.08 | 1.00 | 2.00 | 4.00 | 6.00 | 8.00 | 24.00 |
| Dose Level | | NPT-042 | | | | | | |
| (mg/kg) | Animal | (ng/ml) | | | | | | |
| 10 | 1201A | 5210 | 2000 | 1140 | 471 | 276 | 147 | 9.46 |
| | 1501A | 5900 | 2240 | 1230 | 688 | 326 | 182 | 20.0 |
| | Mean | 5560 | 2120 | 1190 | 580 | 301 | 165 | 14.7 |

TABLE 7

| | | Time | | | | | | |
|---|---|---|---|---|---|---|---|---|
| | | (h) | | | | | | |
| | | 0.08 | 1.00 | 2.00 | 4.00 | 6.00 | 8.00 | 24.00 |
| Dose Level | | Bumetanide | | | | | | |
| (mg/kg) | Animal | (ng/ml) | | | | | | |
| 10 | 1201A | 0.42 | (<0.250) | (<0.250) | (<0.250) | (<0.250) | (<0.250) | (<0.250) |
| | 1501A | 0.34 | 0.29 | (<0.250) | (<0.250) | (<0.250) | (<0.250) | (<0.250) |
| | Mean | 0.377 | 0.288 | | | | | |

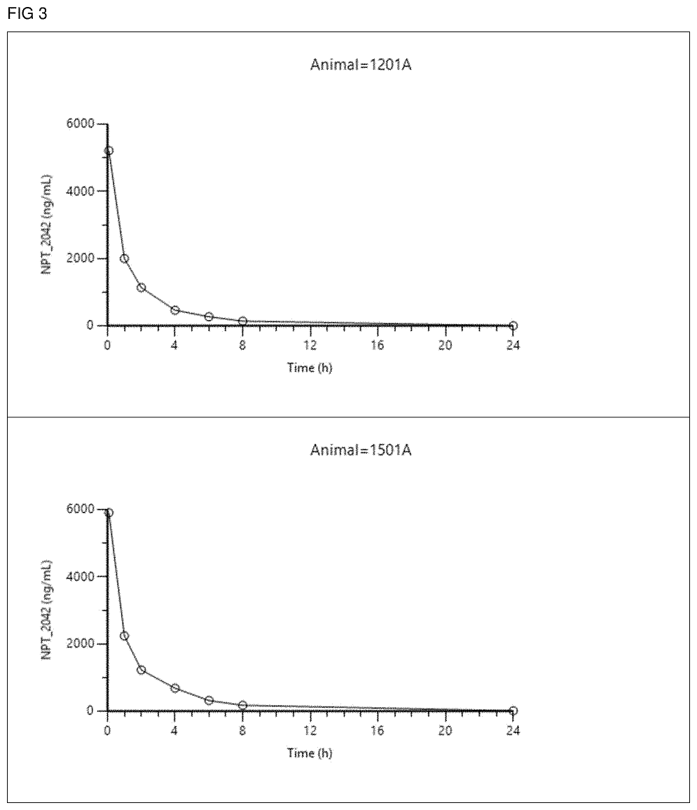

FIG. 3 graphically illustrates plasma NPT-2042 vs Time Plots (linear) for 10 mg/kg Bolus Dosing of NPT-2042 in Dogs (n=2)

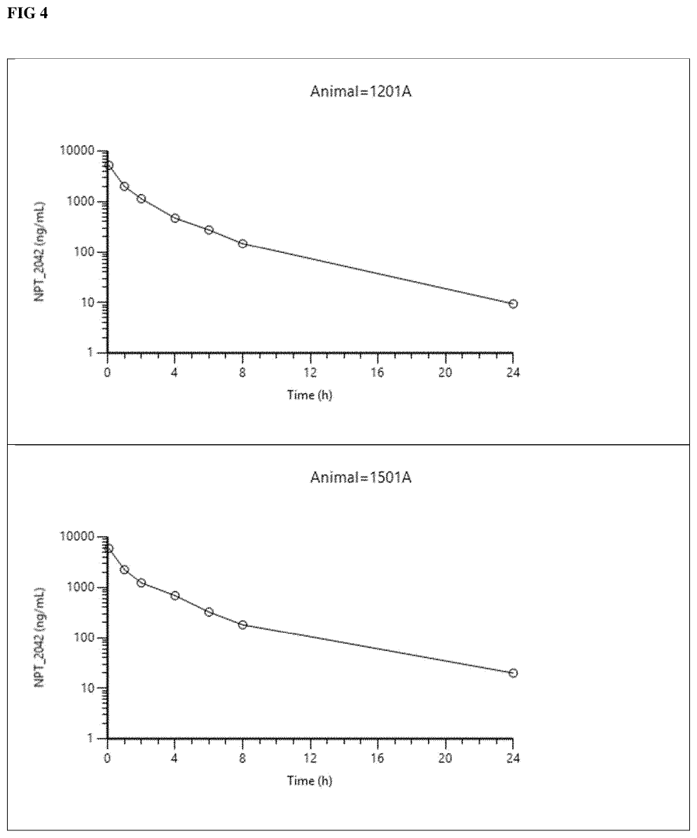

FIG. 4 graphically illustrates plasma NPT-2042 vs Time Plots (log-linear) for 10 mg/kg Bolus Dosing of NPT-2042 in Dogs (n=2)

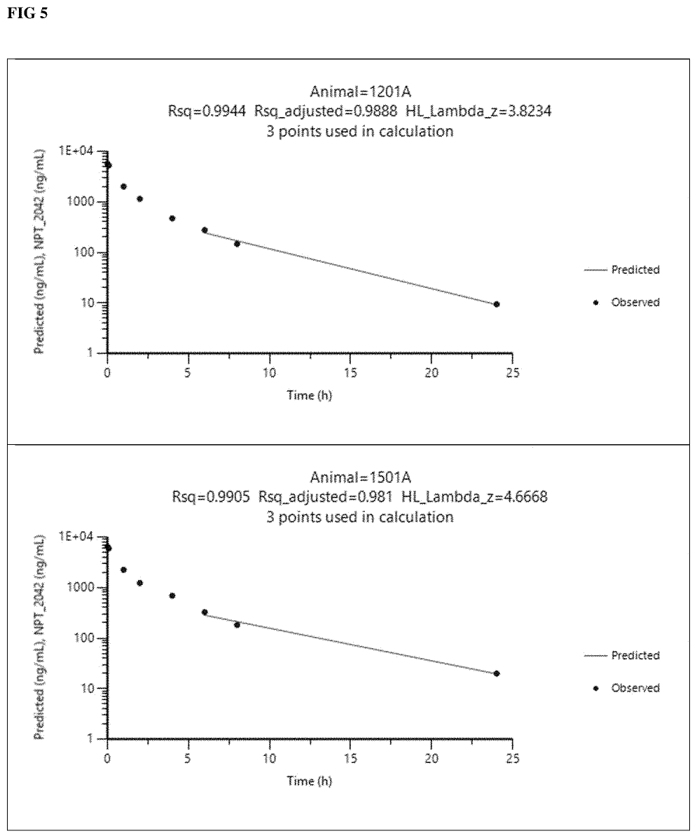

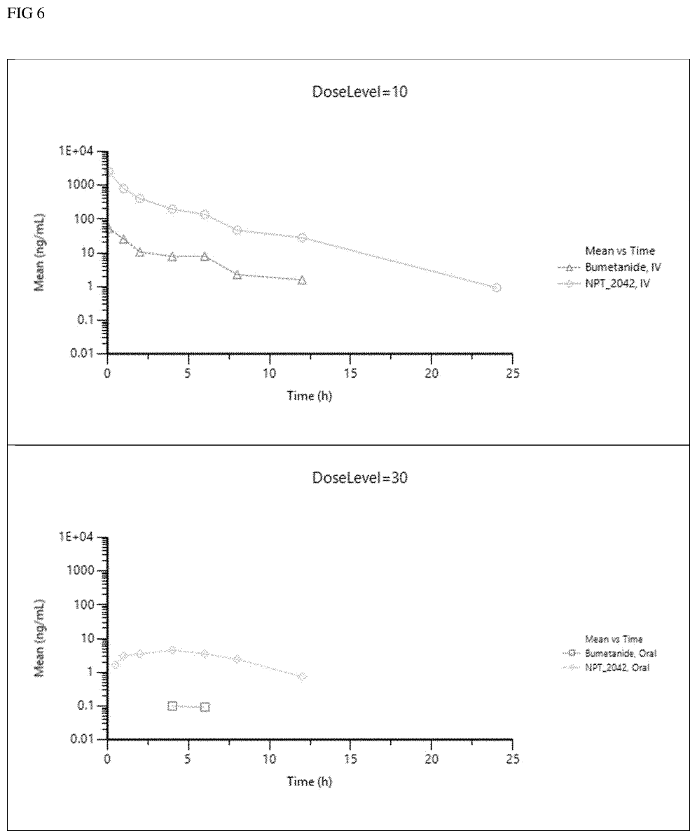

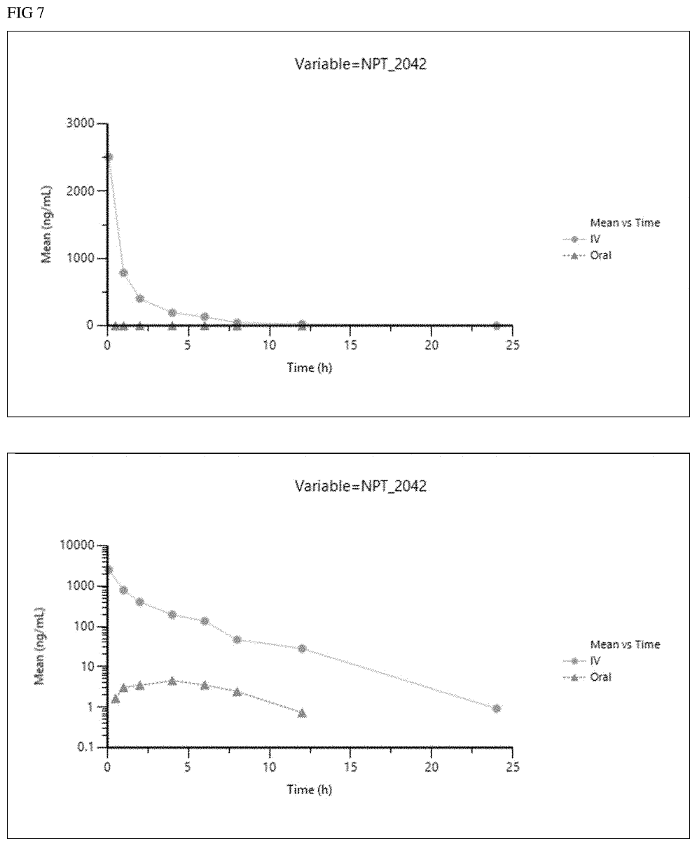

FIG. 5 graphically illustrates WinNonlin Plots for Lambda-z Estimation for 10 mg/kg Bolus Dosing of NPT-2042 in Dogs (n=2)

Table 8 provides Noncompartmental Parameters for NPT-2042 for 10 mg/kg Bolus Dosing of NPT-2042 in Dogs (n=2).

TABLE 8

| Dose | | Cmax | C0 | Tmax | t½ | AUCinf | CL | Vz |
|---|---|---|---|---|---|---|---|---|
| (mg/kg) | Animal | (ng/mL) | (ng/mL) | (h) | (h) | (h * ng/mL) | (L/h/kg) | (L/kg) |
| 10 | 1201A | 5210 | 5680 | 0.08 | 3.82 | 8560 | 1.17 | 6.44 |
| | 1501A | 5900 | 6440 | 0.08 | 4.67 | 10300 | 0.971 | 6.54 |
| | Mean | 5560 | 6060 | 0.0830 | 4.25 | 9430 | 1.07 | 6.49 |

### Example 5: Rat

Testing was conducted to determine AUCs for an oral dose in rat.

TABLE 9
Summary of Individual Rat NPT-2042 Concentration-Time
Data following an Oral Dose of 10 mg/kg NPT-2042

| | | | Time | | | | | | | |
|---|---|---|---|---|---|---|---|---|---|---|
| | | | (h) | | | | | | | |
| | | | 0.50 | 1.00 | 2.00 | 4.00 | 6.00 | 8.00 | 12.00 | 24.00 |
| | | | NPT-2042 | | | | | | | |
| Route | Dose (mg/kg) | Animal | (ng/ml) | | | | | | | |
| Oral | 30 | R0101 | 2.01 | | 4.00 | | 3.99 | | 0.780 | |
| | | R0102 | 1.29 | | 2.88 | | 2.49 | | 0.715 | |
| | | R0103 | 1.67 | | 3.60 | | 4.15 | | 0.731 | |
| | | R0104 | | 2.14 | | 3.52 | | 1.57 | | |
| | | R0105 | | 4.70 | | 5.42 | | 2.57 | | |
| | | R0106 | | 2.45 | | 4.73 | | 3.20 | | |
| | | N | 3 | 3 | 3 | 3 | 3 | 3 | 3 | 0 |
| | | Mean | 1.66 | 3.10 | 3.49 | 4.56 | 3.54 | 2.45 | 0.742 | |
| | | CV % | 21.7 | 45.1 | 16.2 | 21.1 | 25.8 | 33.6 | 4.6 | |

TABLE 10

| | Summary of Individual Rat Bumetanide Concentration- | | | | | | | | | |
|---|---|---|---|---|---|---|---|---|---|---|
| | Time Data following an Oral Dose of 10 mg/kg NPT-2042 | | | | | | | | | |
| | | | Time | | | | | | | |
| | | | (h) | | | | | | | |
| | | | 0.50 | 1.00 | 2.00 | 4.00 | 6.00 | 8.00 | 12.00 | 24.00 |
| | Dose | | Bumetanide | | | | | | | |
| Route | (mg/kg) | Animal | (ng/mL) | | | | | | | |
| Oral | 30 | R0101 | 0.00 | | 0.00 | | 0.00 | | 0.00 | |
| | | R0102 | 0.00 | | 0.00 | | 0.00 | | 0.00 | |
| | | R0103 | 0.00 | | 0.00 | | 0.28 | | 0.00 | |
| | | R0104 | | 0.00 | | 0.00 | | 0.00 | | 0.00 |
| | | R0105 | | 0.00 | | 0.30 | | 0.00 | | 0.00 |
| | | R0106 | | 0.00 | | 0.00 | | 0.00 | | 0.00 |
| | | N | 3 | 3 | 3 | 3 | 3 | 3 | 3 | 3 |
| | | Mean | 0.00 | 0.00 | 0.00 | 0.100 | 0.0920 | 0.00 | 0.00 | 0.00 |
| | | CV % | | | | 173.2 | 173.2 | | | |

Testing was conducted to determine AUCs for an oral dose in rat.

TABLE 11
Summary of Individual Rat NPT-2042 Concentration-Time
Data following an IV Bolus Dose of 10 mg/kg NPT-2042

| | | | Time | | | | | | | |
|---|---|---|---|---|---|---|---|---|---|---|
| | | | (h) | | | | | | | |
| | | | 0.08 | 1.00 | 2.00 | 4.00 | 6.00 | 8.00 | 12.00 | 24.00 |
| | | | NPT-2042 | | | | | | | |
| Route | Dose (mg/kg) | Animal | (ng/ml) | | | | | | | |
| IV | 10 | R0001 | 1720 | | 317 | | 203 | | 60.8 | |
| | | R0002 | 3080 | | 477 | | 145 | | 18.6 | |
| | | R0003 | 2720 | | 421 | | 60.8 | | 4.67 | |
| | | R0004 | | 250 | | 165 | | 20.0 | | 0.257 |
| | | R0005 | | 1190 | | 205 | | 33.1 | | 0.582 |
| | | R0006 | | 924 | | 218 | | 87.3 | | 1.95 |
| | | N | 3 | 3 | 3 | 3 | 3 | 3 | 3 | 3 |
| | | Mean | 2510 | 788 | 405 | 196 | 136 | 46.8 | 28.0 | 0.930 |
| | | CV % | 28.1 | 61.5 | 20.0 | 14.1 | 52.5 | 76.2 | 104.3 | 96.6 |

TABLE 12
Summary of Individual Rat Bumetanide Concentration-Time Data
following an IV Bolus Dose of 10 mg/kg NPT-2042

| | | | Time | | | | | | | |
|---|---|---|---|---|---|---|---|---|---|---|
| | | | (h) | | | | | | | |
| | | | 0.08 | 1.00 | 2.00 | 4.00 | 6.00 | 8.00 | 12.00 | 24.00 |
| | | | Bumetanide | | | | | | | |
| Route | Dose (mg/kg) | Animal | (ng/mL) | | | | | | | |
| IV | 10 | R0001 | 44.5 | | 8.33 | | 9.24 | | 3.48 | |
| | | R0002 | 60.3 | | 13.2 | | 11.6 | | 1.29 | |
| | | R0003 | 55.9 | | 10.5 | | 3.09 | | 0.00 | |
| | | R0004 | | 26.9 | | 7.36 | | 0.967 | | 0.00 |
| | | R0005 | | 24.9 | | 7.07 | | 1.35 | | 0.00 |
| | | R0006 | | 25.2 | | 9.01 | | 4.46 | | 0.00 |
| | | N | 3 | 3 | 3 | 3 | 3 | 3 | 3 | 3 |
| | | Mean | 53.6 | 25.7 | 10.7 | 7.81 | 7.98 | 2.26 | 1.59 | 0.00 |
| | | CV % | 15.2 | 4.2 | 22.9 | 13.4 | 55.1 | 84.8 | 110.6 | |

TABLE 13
Summary Table: PK Parameters for NPT-2042 in Rats
following an IV bolus dose of 10 mg/kg NPT-2042

| | | Dose | Cmax | | | AUCinf | | Vz |
|---|---|---|---|---|---|---|---|---|
| | | (mg/ | (ng/ | Tmax | t½ | (h * | CL | (mL/ |
| Variable | Route | kg) | mL) | (h) | (h) | ng/mL) | (mL/h/kg) | kg) |
| NPT- | IV | 10 | 2510 | 0.08 | 2.61 | 3470 | 2880 | 10800 |
| 2042 | | | | | | | | |

TABLE 14
Summary Table: PK Parameters for Bumetanide in
Rats following an IV bolus dose of 10 mg/kg NPT-2042

| | | Dose | Cmax | Tmax | t½ | AUCinf |
|---|---|---|---|---|---|---|
| Variable | Route | (mg/kg) | (ng/ml) | (h) | (h) | (h * ng/mL) |
| Bumetanide | IV | 10 | 53.6 | 0.08 | 2.91 | 112 |

TABLE 15
Summary Table: PK Parameters for NPT-2042 in
Rats following aa oral dose of 30 mg/kg NPT-2042

| | | Dose | Cmax | Tmax | t½ | AUCinf |
|---|---|---|---|---|---|---|
| Variable | Route | (mg/kg) | (ng/mL) | (h) | (h) | (h * ng/mL) |
| NPT-2042 | Oral | 30 | 4.56 | 4.00 | 2.61 | 35.4 |

TABLE 16
Summary Table: Absolute Bioavailability

| | | Dose | AUCinf | Fabs | Fabs |
|---|---|---|---|---|---|
| Variable | Route | (mg/kg) | (h*ng/mL) | (ratio) | (%) |
| NPT-2042 | Oral | 30 | 35.4 | 0.0034 | 0.34% |
| | IV | 10 | 3470 | | |

Measurable concentrations of NPT-2042 were observed in rats after IV and oral doses of 10 and 30 mg/kg, respectively.

NPT-2042 Cmax averaged 2510 ng/mL at 5 minutes after administration of the IV dose and 4.56 ng/mL at a Tmax of 4 hours following oral dosing.

Measurable concentrations of bumetanide were observed following IV dosing of NPT-2042 with an average Cmax of 53.6 ng/mL at 5 minutes after dose administration.

The bumetanide concentrations were below the lower limit of quantification of the bioanalytical method (0.1 ng/mL) in all but 2 samples following oral administration of 30 mg/kg NPT-2042.

The terminal phase half-lives for NPT-2042 were identical after each route of administration at 2.61 hours. The half-life of bumetanide after IV administration of NPT-2042 was 2.91 hours.

CL and Vz values after IV administration were 2880 mL/h/kg and 10800 mL/kg indicating that NPT-2042 has a high clearance and large volume of distribution in rats.

The absolute bioavailability is very low in the rat with a fraction absorbed of 0.0034 (0.34%) suggesting the possibility of a very high first-pass extraction by the liver.

| Metabolite Screening of NPT-2042 in Rat, Dog, and Human Microsomes | | | | | | | |
|---|---|---|---|---|---|---|---|
| | Molecular | | | | | | |
| | Ions of | | | | | | Human |
| | Tentative | | | Mass | Rat Mic | Dog Mic | Mic |
| | Metabolites | Retention | Tentative | Calculation | (MS | (MS | (MS |
| Metabolites ID | [M + H]* | Time (min) | Biotransformation | (Da) | Intensity) | Intensity) | Intensity) |
| M1 | 398 | 8.98 | De-alkylations | −146 | 1.28E+07 | 2.95E+06 | 1.42E+06 |
| M2 | 470 | 9.23 | Mono-oxidation and | −74 | 6.62E+06 | 6.84E+05 | 4.34E+06 |
| | | | De-alkylation | | | | |
| M3 | 504 | 9.60 | Mono-oxidation and | −40 | 4.14E+06 | 1.09E+06 | 4.05E+05 |
| | | | De-alkylation | | | | |
| M4 | 488 | 11.39 | De-alkylation | −56 | 6.53E+07 | 3.11E+07 | 2.52E+07 |
| M5 | 560 | 11.51 | Mono-oxidation | 16 | 7.91E+06 | 4.39E+06 | 6.46E+06 |
| M6 | 454 | 12.33 | De-alkylation | −90 | 7.96E+07 | 4.12E+07 | 3.25E+07 |
| NPT-2042 | 544 | 15.29 | NA | NA | 7.73E+07 | 1.85E+08 | 1.64E+08 |
| NPT-2042, T = 0 | 544 | | NA | NA | 4.15E+08 | 3.79E+08 | 3.71E+08 |

### Example 6

### Summary:

1) NPT-2042 underwent major biotransformation pathways of De-alkylation, Mono-oxidation, and Mono-oxidation and De-alkylation.
2) A positive control, diclofenac, showed Phase I metabolism.
3) The structure elucidation of metabolites may be performed.

Procedure

Incubation of Test Compound in Microsomes
1. Compound final concentration: 10 uM.
2. Microsomes concentration 1 mg/mL.
3. Incubate for 1 hour, with an NADPH regenerating system.
4. 0.5 mL of incubation mixture at 0 min and 1 hour, mixed with 0.5 mL of acetonitrile.
5. Vortex, and centrifuge at 13200 rpm for 10 min.
6. Supernatant was transferred to a clean vial for LCMS analysis NADPH regenerating system (cofactor; consisting of NADP [0.340 mg/mL], G6P [1.56 mg/mL], and G6PDH [0.4 U/mL] solutions)

| Time | Percentage of |
|---|---|
| (min) | mobile phase B |
| 0 | 3% |
| 10 | 50% |
| 20 | 97% |
| 22 | 97% |
| 22.1 | 3% |
| 30 | 3% |

LC-MS/MS Method:
HPLC Column: Agilent, Polaris 3 C18-A, 150×2.0 mm (S/N: 533819), made in Netherlands.
Guard Column: Agilent, MonoChrom 3 C18 MetaGuard, 10×2.0 mm (S/N: 556145)
Column temperature: 40° C.
Mobile Phase A: 5 mM ammonium format in water (Optima)
Mobile Phase B: acetonitrile (Optima)
Flow rate: 0.2 mL/min
Gradient (Table on right)
MS: Thermo Q Exactive Plus.

| Time | Percentage of |
|---|---|
| (min) | mobile phase B |
| 0 | 3% |
| 5 | 50% |
| 20 | 97% |
| 22 | 97% |
| 22.1 | 3% |
| 30 | 3% |

Summary of Proposed Metabolite Structures and Biotransformation Pathway

Summary of Proposed Metabolite Structures and Biotransformation Pathway

| | | | Proposed | Proposed |
|---|---|---|---|---|
| Metabolite | [M + H]⁺ | Retention | Metabolite | Biotransformation |
| ID | m/z | Time (min) | Structure | Pathway |
| M1 | 398 | 8.98 | 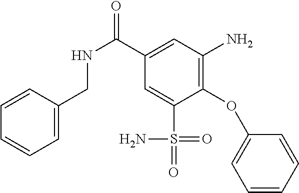 [SMILES: Nc1cc(C(=O)NCc2ccccc2)cc(S(N)(=O)=O)c1Oc1ccccc1] | De-alkylations |
| M2 | 470 | 9.23 | 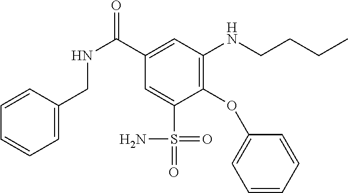 [SMILES: CCCCNc1cc(C(=O)NCc2ccccc2)cc(S(N)(=O)=O)c1Oc1ccccc1] | Mono-oxidation and De- alkylation |
| M3 | 504 | 9.60 | 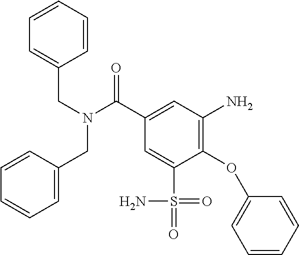 [SMILES: Nc1cc(C(=O)N(Cc2ccccc2)Cc2ccccc2)cc(S(N)(=O)=O)c1Oc1ccccc1] | Mono-oxidation and De- alkylation |
| M4 | 488 | 11.39 | 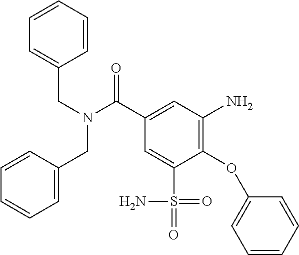 [SMILES: Nc1cc(C(=O)N(Cc2ccccc2)Cc2ccccc2)cc(S(N)(=O)=O)c1Oc1ccccc1] | De-alkylation |
| M5 | 560 | 11.51 | 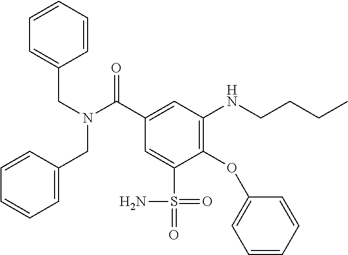 [SMILES: CCCCNc1cc(C(=O)N(Cc2ccccc2)Cc2ccccc2)cc(S(N)(=O)=O)c1Oc1ccccc1] | Mono-oxidation |
| M6 | 454 | 12.33 | 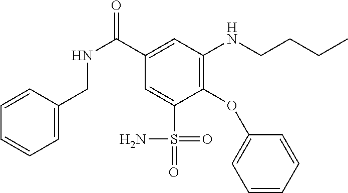 [SMILES: CCCCNc1cc(C(=O)NCc2ccccc2)cc(S(N)(=O)=O)c1Oc1ccccc1] | De-alkylation |
| TA | 544 | 15.29 | 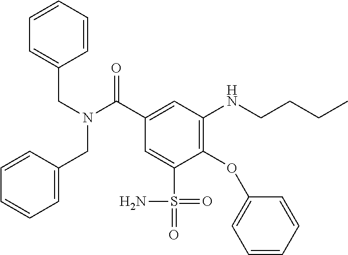 [SMILES: CCCCNc1cc(C(=O)N(Cc2ccccc2)Cc2ccccc2)cc(S(N)(=O)=O)c1Oc1ccccc1] | NA |

### REFERENCES

[1] World Health Organization. Atlas: Epilepsy Care in the World. Geneva: World Health Organization, 2005
[2] Hesdorffer, D. C. Chapter 6: Risk Factors. In Epilepsy: A Comprehensive Text Book. Vol 1. Editors Jerome Engel, Jr. and Timothy A. Pedley. Wolters Kluwer—Lippincott Williams & Wilkins, Philadelphia. 2008. P.57
[3] Berg A T, Shinnar S. The risk of seizure recurrence following a first unprovoked seizure: a quantitative review. Neurology. 1991 July; 41(7):965-72.
[4] Hesdorffer D C, Stables J P, Hauser W A, Annegers J F, Cascino G. Are certain diuretics also anticonvulsants? Ann Neurol. 2001 October; 50(4):458-62.
[5] Sirven J I, Pedley T A, Wilterdink J L (2014). Evaluation and management of drug resistant epilepsy. Available at: http://www.uptodate.com/contents/evaluation-and-management-of-drug-resistant-epilepsy. Accessed September 2015.
[6] Centers for Disease Control and Prevention (2015). Epilepsy Fast Facts. Available at: http://www.cdc.gov/epilepsy/basics/fast-facts.htm. Accessed September 2015.
[7] Begley C E, Famulari M, Annegers J F, et al. The cost of epilepsy in the United States: an estimate from population-based clinical and survey data. Epilepsia 2000; 41:342.
[8] Jöźwiak S, Kotulska K. Prevention of epileptogenesis—a new goal for epilepsy therapy. Pediatr Neurol. 2014 December; 51(6):758-9
[9] U. S. Food and Drug Administration. Suicidal Behavior and Ideation and Antiepileptic Drugs. Available at:http://www.fda.qov/Drugs/DrugSafety/PostmarketDrugSafetylnformationforPatientsandProviders/ucm100190.htm. Accessed September 2015.
[10] Kaufman K R. Antiepileptic drugs in the treatment of psychiatric disorders. Epilepsy Behav. 2011 May; 21(1):1-11.
[11] New York Times. Health Guide: Epilepsy In-Depth Report. Available at: http://www.nytimes.com/health/guides/disease/epilepsy/medications.html. Accessed September, 2015.
[12] Perucca P., Carter J., Vahle V., and Gilliam F. G.: Adverse antiepileptic drug effects: toward a clinically and neurobiologically relevant taxonomy. Neurology 2009; 72: pp. 1223-1229
[13] de Kinderen R J, Evers S M, Rinkens R, Postulart D, Vader C I, Majoie M H, Aldenkamp A P. Side-effects of antiepileptic drugs: the economic burden. Seizure. 2014 March; 23(3):184-90.
[14] Carpay J. A., Aldenkamp A. P., and van Donselaar C. A.: Complaints associated with the use of antiepileptic drugs: results from a community-based study. Seizure 2005; 14: pp. 198-206.
[15] Siniscalchi A, Gallelli L, Russo E, De Sarro G. A review on antiepileptic drugs-dependent fatigue: pathophysiological mechanisms and incidence. Eur J Pharmacol. 2013 Oct. 15; 718(1-3)
[16] Oh, C. Y., and Bainbridge, J. Lowering the seizure threshold associated with antidepressants, stimulants, antipsychotics, and others. Mental Health Clinician. Vol, 2:5, pp.=127-128, 2012
[17] Ijff D M, Aldenkamp A P. Cognitive side-effects of antiepileptic drugs in children. Handb Clin Neurol. 2013; 111:707-18
[18] R. Sankar, G. L. Holmes. Mechanisms of action for the commonly used antiepileptic drugs: relevance to antiepileptic drug-associated neurobehavioral adverse effects. J Child Neurol, 19 (Suppl. 1) (2004), pp. S6—S14
[19] Kobau R., Zahran H., Thurman D. J., et al: Epilepsy surveillance among adults-19 states, Behavioral Risk Factor Surveillance System, 2005. MMWR Surveill Summ 2008; 57: pp. 1-20
[20] Mula M, Trimble M R. Antiepileptic drug-induced cognitive adverse effects: potential mechanisms and contributing factors. CNS Drugs. 2009; 23(2):121-37
[21] Svalheim S, Sveberg L, Mochol M, Taubøll E. Interactions between antiepileptic drugs and hormones. Seizure. 2015 May; 28:12-7.
[22] Chen B, Choi H, Hirsch L J, Moeller J, Javed A, Kato K, Legge A, Buchsbaum R, Detyniecki K. 39. Cosmetic side effects of antiepileptic drugs in adults with epilepsy. Epilepsy Behav. 2015 January; 42:129-37
[23] Walker L E, Mirza N, Yip V L, Marson A G, Pirmohamed M. Personalized medicine approaches in epilepsy. J Intern Med. 2015 February; 277(2):218-34.
[24] I. E. Leppik How to get patients with epilepsy to take their medication. The problem of noncompliance. Postgrad Med, 88 (July (1)) (1990), pp. 253-256
[25] Faught E.: Adherence to antiepilepsy drug therapy. Epilepsy Behav 2012; 25: pp. 297-302.
[26] Faught E., Duh M. S., Weiner J. R., Guérin A., and Cunnington M. C.: Nonadherence to antiepileptic drugs and increased mortality: findings from the RANSOM study. Neurology 2008; 71: pp. 1572-1578
[27] Hamiwka L., Jones J. E., Salpekar J., and Caplan R.: Child psychiatry: special edition on the future of clinical epilepsy research. Epilepsy Behav 2011; 22: pp. 38-46
[28] Lin J. J., Mula M., and Hermann B. P.: Uncovering the neurobehavioural comorbidities of epilepsy over the lifespan. Lancet 2013; 380: pp. 1180-1192
[29]. Adelow C., Andersson T., Ahlbom A., and Tomson T.: Hospitalization for psychiatric disorders before and after onset of unprovoked seizures/epilepsy. Neurology 2012; 78: pp. 396-401
[30]. Hesdorffer D. C., Ludvigsson P., Olafsson E., Gudmundsson G., Kjartansson O., and Hauser W. A.: ADHD as a risk factor for incident unprovoked seizures and epilepsy in children. Arch Gen Psychiatry 2004; 61: pp. 731-736
[31]. Hesdorffer D., Hauser W. A., Olafsson E., Ludvigsson P., and Kjartansson O.: Depression and suicide attempt as risk factors for incident unprovoked seizures. Ann Neurol 2006; 59: pp. 35-41
[32]. Hesdorffer D. C., Ishihara L., Mynepalli L., Webb D. J., Weil J., and Hauser W. A.: Epilepsy, suicidality, and psychiatric disorders: a bidirectional association. Ann Neurol 2012; 72: pp. 184-191
[33]. Ottman R., Lipton R. B., Ettinger A. B., Cramer J. A., Reed M. L., Morrison A., et al: Comorbidities of epilepsy: results from the Epilepsy Comorbidities and Health (EPIC) survey. Epilepsia 2012; 52: pp. 308-315
[34]. Qin P., Xu H., Laursen T. M., Vestergaard M., and Mortensen P. B.: Risk for schizophrenia and schizophrenia-like psychosis among patients with epilepsy: population based cohort study. BMJ 2005; 331: pp. 23
[35]. Wotton C. J., and Goldacre M. J.: Coexistence of schizophrenia and epilepsy: record-linkage studies. Epilepsia 2012; 53: pp. 71-74
[36] Accardo J A, Malow B A. Sleep, epilepsy, and autism. Epilepsy Behav. 2015 June; 47:202-6
[37] Malow B. A., Levy K., Maturen K., and Bowes R.: Obstructive sleep apnea is common in medically refractory epilepsy patients. Neurology 2000; 55: pp. 1002-1007
[38] Woolfenden S, Sarkozy V, Ridley G, Coory M, Williams K. A systematic review of two outcomes in autism spectrum disorder—epilepsy and mortality. Dev Med Child Neurol. 2012 April; 54(4):306-12.
[39] R. Tuchman. Autism and Seizures. Epilepsy Behav, 13 Aug. 2001, Volume 28, Issue 2, Pages 311-311,
[40] Maayan, L., & Correll, C. U. (2011). Weight gain and metabolic risks associated with antipsychotic medications in children and adolescents. Journal of child and adolescent psychopharmacology, 21, 517-535.
[41] Lyngberg A C, Rasmussen B K, Jorgensen T, et al. Incidence of primary headache: a Danish epidemiologic follow-up study. Am J Epidemiol. 2005; 161:1066-1073.
[42] Ottman R, Lipton R B. Comorbidity of migraine and epilepsy. Neurology. 1994; 44:2105-2110.
[43] Velioglu S K, Boz C, Ozmenoglu M. The impact of migraine on epilepsy: a prospective prognosis study. Cephalalgia. 2005; 25:528-535.
[44] Breslau N, Davis G C. Migraine, physical health and psychiatric disorder: a prospective epidemiologic study in young adults. J Psychiatr Res. 1993; 27:211-221.
[45] Hesdorffer D C, Ludvigsson P, Hauser W A, et al. Co-occurrence of major depression or suicide attempt with migraine with aura and risk for unprovoked seizure. Epilepsy Res. 2007; 75:220-223.
[46] Wilner A N, Sharma B K, Soucy A, Thompson A, Krueger A. Common comorbidities in women and men with epilepsy and the relationship between number of comorbidities and health plan paid costs in 2010. Epilepsy Behav. 2014 March; 32:15-20.
[47] Lambert, M., Robertson, M. Depression in epilepsy: etiology, phenomenology, and treatment. Epilepsia 40, 1999, Suppl. 10, S21-47.
[48] Harden, C., Goldstein, M. Mood disorders in patients with epilepsy: epidemiology and management. CNS Drugs, 16, 2002, 291-302.
[49] Dobesberger J, Ristic A2, Walser G, Höfler J4, Unterberger I, Trinka E. Selective serotonin reuptake inhibitors prolong seizures—preliminary results from an observational study. Clin Neurol Neurosurg. 2014 May; 120:89-92
[50] Jackson M J, Turkington D. Depression and anxiety in epilepsy. J Neurol Neurosurg Psychiatry. 2005 March; 76 Suppl 1:i45-47.
[51] Kwon O Y, Park S P. Depression and anxiety in people with epilepsy. J Clin Neurol. 2014 July; 10(3):175-88.
[52] Torta R, Keller R. Behavioral, psychotic, and anxiety disorders in epilepsy: etiology, clinical features, and therapeutic implications. Epilepsia. 1999. 40 Suppl 10:S2-20
[53] Ettinger A B, Ottman R, Lipton R B, Cramer J A, Fanning K M, Reed M L. Attention-deficit/hyperactivity disorder symptoms in adults with self-reported epilepsy: Results from a national epidemiologic survey of epilepsy. Epilepsia. 2015 February; 56(2):218-24.
[54]. Dunn D. W., Austin J. K., Harezlak J., and Ambrosius W. T.: ADHD and epilepsy in childhood. Dev Med Child Neurol 2003; 45: pp. 50-54
[55]. Hermann B., Jones J., Dabbs K., et al: The frequency, complications and aetiology of ADHD in new onset paediatric epilepsy. Brain 2007; 130: pp. 3135-3148
[56]. Davies S., Heyman I., and Goodman R.: A population survey of mental health problems in children with epilepsy. Dev Med Child Neurol 2003; 45: pp. 292-295
[57]. Hesdorffer D. C., Ludvigsson P., Olafsson E., Gudmundsson G., Kjartansson O., and Hauser W. A.: ADHD as a risk factor for incident unprovoked seizures and epilepsy in children. Arch Gen Psychiatry 2004; 61: pp. 731-736
[58]. Ishii T., Takahashi O., Kawamura Y., and Ohta T.: Comorbidity in attention deficit-hyperactivity disorder. Psychiatry Clin Neurosci 2003; 57: pp. 457-463
[59]. Williams J., Schulz E. G., and Griebel M. L.: Seizure occurrence in children diagnosed with ADHD. Clin Pediatr (Phila) 2001; 40: pp. 221-224
[60]. Wisśniewska B., Baranowska W., and Wendorff J.: The assessment of comorbid disorders in ADHD children and adolescents. Adv Med Sci 2007; 52: pp. 215-217
[61]. Katusic S. K., Barbaresi W. J., Colligan R. C., Weaver A. L., Leibson C. L., and Jacobsen S. J.: Case definition in epidemiologic studies of AD/HD. Ann Epidemiol 2005; 15: pp. 430-437
[62] Harnod T, Chen H J, Li T C, Sung F C, Kao C H. A high risk of hyperlipidemia in epilepsy patients: a nationwide population-based cohort study. Ann Epidemiol. 2014 December; 24(12):910-4.
[63] Michael Seidenberg, Dalin T Pulsipher, Bruce Hermann. Association of epilepsy and comorbid conditions. Future Neurol. 2009 September 1; 4(5): 663-668.
[64] Epilepsy Foundation. Seizures and Headaches. Available at: http://www.epilepsy.com/article/2014/3/seizures-and-headaches-they-dont-have-go-together. Last accessed: September 2015.
[65] Epilepsy Foundation. Types of epilepsy syndromes. Available at: http://www.epilepsy.com/learn/types-epilepsy-syndromes. Accessed September 2015.
[66] Deb, S., Epilepsy and mental retardation. Epilepsie Bulletin, 25, 91-94. 1997.
[67] Stafstrom, CE. Epilepsy in Down Syndrome: Clinical aspects and possible mechanisms. Am J Mental Retard 98: S12-26, 1993.
[68] Friedman D L, Kastner T, Plummer A T. Adverse behavioural effects in individuals with mental retardation and mood disorders treated with carbamazepine. Am J Ment Retard. 1992; 96:541-546.
[69] Bourgeois, B. F. E, Gilliam, F. Chapter 121: Adjunctive and Combination Therapy. In Epilepsy: A Comprehensive Text Book. Vol 1. Editors Jerome Engel, Jr. and Timothy A. Pedley. Wolters Kluwer—Lippincott Williams & Wilkins, Philadelphia. 2008. P. 57
[70] Witt J A, Elger C E, Helmstaedter C Adverse cognitive effects of antiepileptic pharmacotherapy: Each additional drug matters. Eur Neuropsychopharmacol. 2015 Aug. 6. pii: 50924-977X(15)00245-X. doi: 10.1016/j.euroneuro.2015.07.027. [Epub ahead of print]
[71] Ahmad S, Clarke L, Hewett A J, Richens A. (1976) Controlled trial of frusemide as an antiepileptic drug in focal epilepsy. Br J Clin Pharmacol 3:621-625.
[72] Haglund M M, Hochman D W. (2005) Furosemide and mannitol suppression of epileptic activity in the human brain. J Neurophysiol 94:907-918.
[73] Hochman D W, Baraban S C, Owens J W, Schwartzkroin P A. (1995) Dissociation of synchronization and excitability in furosemide blockade of epileptiform activity. Science 270:99-102
[74] Eftekhari 51, Mehvari Habibabadi J, Najafi Ziarani M, Hashemi Fesharaki S S, Gharakhani M, Mostafavi H, Joghataei M T, Beladimoghadam N, Rahimian E, Hadjighassem M R. Bumetanide reduces seizure frequency in patients with temporal lobe epilepsy. Epilepsia. 2013 January; 54(1):e9-el 2.
[75] Krystal A D, Sutherland J, Hochman D W. Loop diuretics have anxiolytic effects in rat models of conditioned anxiety. PLoS One. 2012; 7(4):e35417.
[76] Holmes G L, Tian C, Hernan A E, Flynn S, Camp D, Barry J. Neurobiol Dis. 2015 May; 77:204-19.
[77] Lemonnier El, Degrez C, Phelep M, Tyzio R, Josse F, Grandgeorge M, Hadjikhani N, Ben-Ari Y. A randomised controlled trial of bumetanide in the treatment of autism in children. Transl Psychiatry. 2012 Dec. 11; 2:e202
[78] Rozen T D. Treatment of a prolonged migrainous aura with intravenous furosemide. Neurology. 2000 Sep. 12; 55(5):732-3
[79] Read S J, Smith M I, Benham C D, Hunter A J, Parsons A A. Furosemide inhibits regenerative cortical spreading depression in anaesthetized cats. Cephalalgia. 1997 December; 17(8):826-32.
[80] Mathew N T, Ravishankar K, Sanin L C. Coexistence of migraine and idiopathic intracranial hypertension without papilledema. Neurology. 1996 May; 46(5):1226-30
[81] Mulders W H, McMahen C, Robertson D. Effects of chronic furosemide on central neural hyperactivity and cochlear thresholds after cochlear trauma in Guinea pig. Front Neurol. 2014 Aug. 8; 5:146.
[82] Hasbargen T, Ahmed M M, Miranpuri G, Li L, Kahle K T, Resnick D, Sun D. Role of NKCC1 and KCC2 in the development of chronic neuropathic pain following spinal cord injury. Ann N Y Acad Sci. 2010 June; 1198:168-72
[83] Deidda G, Parrini M, Naskar S, Bozarth I F, Contestabile A, Cancedda L. Reversing excitatory GABAAR signaling restores synaptic plasticity and memory in a mouse model of Down syndrome. Nat Med. 2015 April; 21(4):318-26.
[84] Töllner K, Brandt C, Töpfer M, Brunhofer G, Erker T, Gabriel M, Feit P W, Lindfors J, Kaila K, Loscher W. A novel prodrug-based strategy to increase effects of bumetanide in epilepsy. Ann Neurol. 2014 April; 75(4):550-62.

Those skilled in the art to which the present disclosure pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present disclosure is, therefore, indicated by the appended claims rather than by the foregoing description or drawings. Consequently, while the present invention has been described with reference to particular embodiments, modifications of structure, sequence, materials and the like apparent to those skilled in the art still fall within the scope as claimed.

What is claimed is:

1. A pharmaceutical composition comprising:
i) an active agent selected from one or more of bumetanide morpholinoamide, bumetanide diethylamide, and bumetanide dibenzylamide, or a salt thereof; and
ii) one or more excipient to increase bioavailability of the one or more active agent.

2. The pharmaceutical composition of claim 1, wherein the active agent is bumetanide dibenzylamide.

3. A pharmaceutical composition comprising:
i) an active agent bumetanide dibenzylamide, or a salt thereof; and
ii) one or more excipient,
wherein the minimum area under curve is 57 ng*h/mL for each immediate release formulated dose, and a minimum total AUC of 114 ng*h/mL over a 24 hr period with a sustained release formulated dose.

4. A pharmaceutical composition comprising:
iii) an active agent bumetanide morpholinoamide, or a salt thereof; and
iv) one or more excipient,
wherein the minimum area under curve is 45 ng*h/mL for each immediate release formulated dose, and a minimum total AUC of 84 ng*h/mL over a 24 hr period with a sustained release formulated dose.

5. A pharmaceutical composition comprising:
v) an active agent bumetanide diethylamide, or a salt thereof; and
vi) one or more excipient,
wherein the minimum area under curve is 42 ng*h/mL for each immediate release formulated dose, and a minimum total AUC of 84 ng*h/mL over a 24 hr period with a sustained release formulated dose.

6. The pharmaceutical composition of any one of claims 1-5, wherein the composition is selected from the group consisting of: a sublingual tablet, a sublingual amorphous solid dispersion, an orodispersible film, a fast dissolving film, a nanoparticle spray, a muco-adhesion patch, a liquid, a slick pack, a lipid-filled soft gelatin capsule, a liquid-filled hard gelatin capsule, a transdermal patch, a transdermal gel, a nasal drop, a nasal spray, a nasal powder, a nasal gel, a suppository, a rectal solution, a subcutaneous solution or emulsion, an intravenous solution or emulsion, an intramuscular injectable, intramuscular microspheres, intramuscular hydrogels, intramuscular organogels, intramuscular liquid crystals, an intramuscular solution, an inhalation solution, and an inhalation powder.

7. The pharmaceutical composition of any one of claims 3-5, wherein a minimum Cmax is 25 ng/mL.

8. The pharmaceutical composition of any one of claims 3-5, wherein a minimum Cmax is 20 ng/mL.

9. The pharmaceutical composition of any one of claims 3-5, wherein a minimum Cmax is 18 ng/mL.

10. The pharmaceutical composition of any one of claims 1-9, wherein the composition provides an extended release profile of the active agent of 1 week, 2 weeks, 3 weeks, or 4 weeks.

11. A method of using the pharmaceutical composition to treat one or more disorder susceptible to modulation of NKCC, comprising administering a composition of any one of claims 1 to 10.

12. A method of treating one or more seizure disorders, comprising administering a composition of any one of claims 1 to 10.

13. A method of treating one or more epilepsies, comprising administering a composition of any one of claims 1 to 10.

14. A method of treating one or more epileptic syndromes, comprising administering a composition of any one of claims 1 to 10.

15. A method of treating one or more of Localization-related (focal, local, partial) epilepsies and syndromes, Idiopathic with age-related onset, Benign childhood epilepsy with centrotemporal spikes, Childhood epilepsy with occipital paroxysms, Symptomatic epilepsies, Chronic progressive epilepsia partialis continua of childhood, Syndromes characterized by seizures with specific modes of precipitation, Temporal lobe epilepsies, Frontal lobe epilepsies, Parietal lobe epilepsies, Occipital lobe epilepsies, Crytopgenic epilepsies, Generalized epilepsies and syndromes, Idiopathic, with age-related onset, Benign neonatal familial convulsions, Benign neonatal convulsions, Benign myoclonic epilepsy in infancy, Childhood absence epilepsy (pyknolepsy), Juvenile absence epilepsy, Juvenile myoclonic epilepsy (impulsive petit mal), Epilepsy with grand mal seizures on awakening, Epilepsies with seizures precipitated by specific modes of activation, Idiopathic and/or symptomatic, West syndrome (infantile spasms), Lennox-Gastaut syndrome, Epilepsy with myoclonic-astatic seizures, Epilepsy with myoclonic absences, Symptomatic, Nonspecific etiology epilepsies, Early myoclonic encephalopathy, Early infantile epileptic encephalopathy with suppression burst, Specific etiology epilepsies, Epilepsies and syndromes undetermined as to whether they are focal or generalized, Epilepsies and syndromes with generalized and focal seizures, Neonatal seizures, Severe myoclonic epilepsy in infancy, Epilepsy with continuous spike waves during slow-wave sleep, Acquired epileptic aphasia (Landau-Kleffner syndrome), Other undetermined epilepsies not defined above, Epilepsies and syndromes without unequivocal generalized or focal features, Special syndromes, Situation-related seizures, Febrile convulsions, Isolated, apparently unprovoked epileptic events, and Seizures related to other identifiable situations such as stress, hormonal changes, drugs, alcohol, or sleep deprivation, comprising administering a composition of any one of claims 1 to 10.

16. A combination comprising a pharmaceutical composition of any one of claims 1 to 10 and one or more additional therapeutic agent.

17. The method of any one of claims 11 to 15, further comprising administration of one or more additional therapeutic agent.
